SEQUENCE LISTING (211 sequences)

SEQ ID NO: 1 - protein, 78 aa, synthetic construct
  source 1..78: note = OLP HIV clade B consensus sequence
EKIRLRPGGKKKYKLKHIVWASRELERFAVNPGLLETSEGCRQILGQLQPSLQTGSEELKSLYNTVATLYCVHQKIEV

SEQ ID NO: 2 - protein, 14 aa, synthetic construct
  source 1..14: note = OLP HIV clade B consensus sequence
KAFSPEVIPMFSAL

SEQ ID NO: 3 - protein, 11 aa, synthetic construct
  source 1..11: note = OLP HIV clade B consensus sequence
GHQAAMQMLKE

SEQ ID NO: 4 - protein, 60 aa, synthetic construct
  source 1..60: note = OLP HIV clade B consensus sequence
IAPGQMREPRGSDIAGTTSTLQEQIGWMTNNPPIPVGEIYKRWIILGLNKIVRMYSPTSI

SEQ ID NO: 5 - protein, 14 aa, synthetic construct
  source 1..14: note = OLP HIV clade B consensus sequence
YVDRFYKTLRAEQA

SEQ ID NO: 6 - protein, 15 aa, synthetic construct
  source 1..15: note = OLP HIV clade B consensus sequence
ACQGVGGPGHKARVL

SEQ ID NO: 7 - protein, 27 aa, synthetic construct
  source 1..27: note = OLP HIV clade B consensus sequence
CTERQANFLGKIWPSHKGRPGNFLQSR

SEQ ID NO: 8 - protein, 55 aa, synthetic construct
  source 1..55: note = OLP HIV clade B consensus sequence
KMIGGIGGFIKVRQYDQILIEICGHKAIGTVLVGPTPVNIIGRNLLTQIGCTLNF

SEQ ID NO: 9 - protein, 17 aa, synthetic construct
  source 1..17: note = OLP HIV clade B consensus sequence
LVEICTELEKEGKISKI

SEQ ID NO: 10 - protein, 55 aa, synthetic construct
  source 1..55: note = OLP HIV clade B consensus sequence
LRWGFTTPDKKHQKEPPFLWMGYELHPDKWTVQPIVLPEKDSWTVNDIQKLVGKL

SEQ ID NO: 11 - protein, 34 aa, synthetic construct
  source 1..34: note = OLP HIV clade B consensus sequence
ILKEPVHGVYYDPSKDLIAEIQKQGQGQWTYQIY

SEQ ID NO: 12 - protein, 34 aa, synthetic construct
  source 1..34: note = OLP HIV clade B consensus sequence
TKELQKQITKIQNFRVYYRDSRDPLWKGPAKLLW

SEQ ID NO: 13 - protein, 17 aa, synthetic construct
  source 1..17: note = OLP HIV clade B consensus sequence
KIIRDYGKQMAGDDCVA

SEQ ID NO: 14 - protein, 26 aa, synthetic construct
  source 1..26: note = OLP HIV clade B consensus sequence
VKHHMYISKKAKGWFYRHHYESTHPR

SEQ ID NO: 15 - protein, 19 aa, synthetic construct
  source 1..19: note = OLP HIV clade B consensus sequence
VTKLTEDRWNKPQKTKGHR

SEQ ID NO: 16 - protein, 13 aa, synthetic construct
  source 1..13: note = OLP HIV clade B consensus sequence
AWLEAQEEEEVGF

SEQ ID NO: 17 - protein, 23 aa, synthetic construct
  source 1..23: note = OLP HIV clade B consensus sequence
EKIRLRPGGKKKYKLKHIVWASR

SEQ ID NO: 18 - protein, 22 aa, synthetic construct
  source 1..22: note = OLP HIV clade B consensus sequence
KLKHIVWASRELERFAVNPGLL

SEQ ID NO: 19 - protein, 23 aa, synthetic construct
  source 1..23: note = OLP HIV clade B consensus sequence
ERFAVNPGLLETSEGCRQILGQL

SEQ ID NO: 20 - protein, 24 aa, synthetic construct
  source 1..24: note = OLP HIV clade B consensus sequence
EGCRQILGQLQPSLQTGSEELKSL

SEQ ID NO: 21 - protein, 26 aa, synthetic construct
  source 1..26: note = OLP HIV clade B consensus sequence
QTGSEELKSLYNTVATLYCVHQRIEV

SEQ ID NO: 22 - protein, 14 aa, synthetic construct
  source 1..14: note = OLP HIV clade B consensus sequence
KAFSPEVIPMFSAL

SEQ ID NO: 23 - protein, 11 aa, synthetic construct
  source 1..11: note = OLP HIV clade B consensus sequence
GHQAAMQMLKE

SEQ ID NO: 24 - protein, 21 aa, synthetic construct
  source 1..21: note = OLP HIV clade B consensus sequence
IAPGQMREPRGSDIAGTTSTL

SEQ ID NO: 25 - protein, 23 aa, synthetic construct
  source 1..23: note = OLP HIV clade B consensus sequence
SDIAGTTSTLQEQIGWMTNNPPI

SEQ ID NO: 26 - protein, 24 aa, synthetic construct
  source 1..24: note = OLP HIV clade B consensus sequence
IGWMTNNPPIPVGEIYKRWIILGL

SEQ ID NO: 27 - protein, 22 aa, synthetic construct
  source 1..22: note = OLP HIV clade B consensus sequence
IYKRWIILGLNKIVRMYSPTSI

SEQ ID NO: 28 - protein, 14 aa, synthetic construct
  source 1..14: note = OLP HIV clade B consensus sequence
YVDRFYKTLRAEQA

SEQ ID NO: 29 - protein, 15 aa, synthetic construct
  source 1..15: note = OLP HIV clade B consensus sequence
ACQGVGGPGHKARVL

SEQ ID NO: 30 - protein, 27 aa, synthetic construct
  source 1..27: note = OLP HIV clade B consensus sequence
CTERQANFLGKIWPSHKGRPGNFLQSR

SEQ ID NO: 31 - protein, 26 aa, synthetic construct
  source 1..26: note = OLP HIV clade B consensus sequence
KMIGGIGGFIKVRQYDQILIEICGHK

SEQ ID NO: 32 - protein, 24 aa, synthetic construct
  source 1..24: note = OLP HIV clade B consensus sequence
QILIEICGHKAIGTVLVGPTPVNI

SEQ ID NO: 33 - protein, 25 aa, synthetic construct
  source 1..25: note = OLP HIV clade B consensus sequence
VLVGPTPVNIIGRNLLTQIGCTLNF

SEQ ID NO: 34 - protein, 17 aa, synthetic construct
  source 1..17: note = OLP HIV clade B consensus sequence
LVEICTEMEKEGKISKI

SEQ ID NO: 35 - protein, 25 aa, synthetic construct
  source 1..25: note = OLP HIV clade B consensus sequence
LRWGFTTPDKKHQKEPPFLWMGYEL

SEQ ID NO: 36 - protein, 25 aa, synthetic construct
  source 1..25: note = OLP HIV clade B consensus sequence
PPFLWMGYELHPDKWTVQPIVLPEK

SEQ ID NO: 37 - protein, 25 aa, synthetic construct
  source 1..25: note = OLP HIV clade B consensus sequence
TVQPIVLPEKDSWTVNDIQKLVGKL

SEQ ID NO: 38 - protein, 23 aa, synthetic construct
  source 1..23: note = OLP HIV clade B consensus sequence
ILKEPVHGVYYDPSKDLIAEIQK

SEQ ID NO: 39 - protein, 21 aa, synthetic construct
  source 1..21: note = OLP HIV clade B consensus sequence
SKDLIAEIQKQGQGQWTYQIY

SEQ ID NO: 40 - protein, 22 aa, synthetic construct
  source 1..22: note = OLP HIV clade B consensus sequence
TKELQKQITKIQNFRVYYRDSR

SEQ ID NO: 41 - protein, 22 aa, synthetic construct
  source 1..22: note = OLP HIV clade B consensus sequence
NFRVYYRDSRDPLWKGPAKLLW

SEQ ID NO: 42 - protein, 17 aa, synthetic construct
  source 1..17: note = OLP HIV clade B consensus sequence
KIIRDYGKQMAGDDCVA

SEQ ID NO: 43 - protein, 26 aa, synthetic construct
  source 1..26: note = OLP HIV clade B consensus sequence
VKHHMYISKKAKGWFYRHHYESTHPR

SEQ ID NO: 44 - protein, 19 aa, synthetic construct
  source 1..19: note = OLP HIV clade B consensus sequence
VTKLTEDRWNKPQKTKGHR

SEQ ID NO: 45 - protein, 13 aa, synthetic construct
  source 1..13: note = OLP HIV clade B consensus sequence
AWLEAQEEEEVGF

SEQ ID NO: 46 - protein, 17 aa, Homo sapiens
MWLQSLLLLGTVACSIS

SEQ ID NO: 47 - protein, 18 aa, synthetic construct
  source 1..18: note = human GM-CSF signal peptide + V
MWLQSLLLLGTVACSISV

SEQ ID NO: 48 - protein, 9 aa, synthetic construct
  source 1..9: note = Flag-tag
DYKDDDDKL

SEQ ID NO: 49 - protein, 556 aa, synthetic construct
  source 1..556: note = HIV immunogen comprising OLPs
MWLQSLLLLGTVACSISVEKIRLRPGGKKKYKLKHIVWASRELERFAVNPGLLETSEGCRQILGQLQPSLQTGSEELKSLYNTVATLYCVHQKIEVAAAKAFSPEVIPMFSALAAAGHQAAMQMLKEAAAIAPGQMREPRGSDIAGTTSTLQEQIGWMTNNPPIPVGEIYKRWIILGLNKIVRMYSPTSIAAAYVDRFYKTLRAEQAAACQGVGGPGHKARVLAAACTERQANFLGKIWPSHKGRPGNFLQSRAAAKMIGGIGGFIKVRQYDQILIEICGHKAIGTVLVGPTPVNIIGRNLLTQIGCTLNFAALVEICTEMEKEGKISKIAAALRWGFTTPDKKHQKEPPFLWMGYELHPDKWTVQPIVLPEKDSWTVNDIQKLVGKLAAAILKEPVHGVYYDPSKDLIAEIQKQGQGQWTYQIYAAATKELQKQITKIQNFRVYYRDSRDPLWKGPAKLLWAAAKIIRDYGKQMAGDDCVAAAVKHHMYISKKAKGWFYRHHYESTHPRAAAVTKLTEDRWNKPQKTKGHRAAAWLEAQEEEEVGFDYKDDDDKL

SEQ ID NO: 50 - DNA, 1674 bp, synthetic construct
  source 1..1674: mol_type = other DNA; note = polynucleotide encoding immunogen comprising HIV OLPs
atgtggctccagagcctgctactcctggggacggtggcctgcagcatctcggtcgagaagatccggctgcggccaggcggaaagaagaagtacaagctgaagcacatcgtctgggcctcgagggagctggagcggttcgcggtgaacccgggacttctggagacgtcggaggggtgcaggcagatcctcggccagctgcagccctctctgcaaacggggtctgaggagctgaagagcctgtacaacacggtggcgaccctctactgcgtccaccagaagatcgaggtggcagcggccaaggcgttctcgccggaggtcatccccatgttctcggcgctggcagctgccggacaccaggccgcgatgcagatgctgaaggaggccgctgcgatcgcaccgggccagatgagggagccacgcggttccgacatcgcgggaaccacctcgacgctccaggagcagatcggatggatgacgaacaacccgccaatcccggtcggggagatctacaagcggtggatcatcctcgggctgaacaagatcgtccggatgtacagcccgacgtcgatcgctgcggcatacgttgaccggttctacaagaccctgagggccgagcaggcagcggcctgccagggggtcggtggaccagggcacaaggcccgagtgctcgcggccgcatgcacggagcggcaggcgaacttcctggggaagatctggccgtcgcacaagggccgaccgggaaacttcctccagtctcgcgcagcggctaagatgatcggaggcatcggaggcttcatcaaagtccgtcagtacgaccagatcctcatcgagatctgcgggcacaaggcgatcggaaccgtgctcgtcggcccaacgcccgtgaacatcatcggccgcaacctgttaacgcagatcggctgcaccctcaacttcgccgcactagtggagatctgcacggagatggagaaggagggcaagatatcgaagatcgcggcagctctgaggtggggcttcaccacgccggacaagaagcaccagaaggagccgccattcctgtggatgggatacgagctgcacccggacaagtggaccgtgcagcccatcgtcctgccggagaaggactcgtggacggtgaacgacatccagaagctcgtggggaagctggcggcagccatcctcaaggagcccgtccacggggtgtactacgacccctctaaggacctgatcgcggagatccagaagcaggggcagggtcagtggacctaccagatctacgcagcagcaaccaaggagctgcagaagcagatcacgaagatccagaacttccgcgtatactaccgcgactcgcgggaccccctgtggaagggccctgcgaagcttctctgggcagccgcgaagatcatccgggactacggcaagcagatggcgggcgacgactgcgtggccgcagcggtgaagcaccatatgtacatctcgaagaaggcgaagggctggttctacagacaccactacgagtccacccaccccagggcagctgcggtgacgaagctgacggaggaccggtggaacaagccccagaagacgaagggtcaccgggcggctgcatggctggaggctcaggaggaggaggaggtgggcttcgattacaaggacgatgacgacaagctgtgataa

SEQ ID NO: 51 - DNA, 60 bp, synthetic construct
  source 1..60: mol_type = other DNA; note = 198HPlus primer
gtcaccgggcggctgcatggctggaggctcaggaggaggaggaggtgggcttctgataag

SEQ ID NO: 52 - DNA, 59 bp, synthetic construct
  source 1..59: mol_type = other DNA; note = 298HMinus primer
aattcttatcagaagcccacctcctcctcctcctgagcctccagccatgcagccgcccg

SEQ ID NO: 53 - protein, 18 aa, synthetic construct
  source 1..18: note = OLP clade B consensus sequence
EKIRLRPGGKKKYKLKHI

SEQ ID NO: 54 - protein, 15 aa, synthetic construct
  source 1..15: note = OLP clade B consensus sequence
ASRELERFAVNPGLL

SEQ ID NO: 55 - protein, 17 aa, synthetic construct
  source 1..17: note = OLP clade B consensus sequence
ERFAVNPGLLETSEGCR

SEQ ID NO: 56 - protein, 17 aa, synthetic construct
  source 1..17: note = OLP clade B consensus sequence
QLQPSLQTGSEELRSLY

SEQ ID NO: 57 - protein, 18 aa, synthetic construct
  source 1..18: note = OLP clade B consensus sequence
SLYNTVATLYCVHQRIEV

SEQ ID NO: 58 - protein, 17 aa, synthetic construct
  source 1..17: note = OLP clade B consensus sequence
AFSPEVIPMFSALSEGA

SEQ ID NO: 59 - protein, 15 aa, synthetic construct
  source 1..15: note = OLP clade B consensus sequence
IAPGQMREPRGSDIA

SEQ ID NO: 60 - protein, 18 aa, synthetic construct
  source 1..18: note = OLP clade B consensus sequence
STLQEQIGWMTNNPPIPV

SEQ ID NO: 61 - protein, 18 aa, synthetic construct
  source 1..18: note = OLP clade B consensus sequence
ACQGVGGPGHKARVLAEA

SEQ ID NO: 62 - protein, 18 aa, synthetic construct
  source 1..18: note = OLP clade B consensus sequence
GKIWPSHKGRPGNFLQSR

SEQ ID NO: 63 - protein, 18 aa, synthetic construct
  source 1..18: note = OLP clade B consensus sequence
WLEAQEEEEVGFPVRPQV

SEQ ID NO: 64 - protein, 18 aa, synthetic construct
  source 1..18: note = OLP clade B consensus sequence
EVGFPVRPQVPLRPMTYK

SEQ ID NO: 65 - protein, 18 aa, synthetic construct
  source 1..18: note = OLP clade B consensus sequence
NYTPGPGIRYPLTFGWCF

SEQ ID NO: 66 - protein, 15 aa, synthetic construct
  source 1..15: note = OLP clade B consensus sequence
RYPLTFGWCFKLVPV

SEQ ID NO: 67 - protein, 17 aa, synthetic construct
  source 1..17: note = OLP clade B consensus sequence
SLHGMDDPEKEVLVWKF

SEQ ID NO: 68 - protein, 18 aa, synthetic construct
  source 1..18: note = OLP clade B consensus sequence
KMIGGIGGFIKVRQYDQI

SEQ ID NO: 69 - protein, 18 aa, synthetic construct
  source 1..18: note = OLP clade B consensus sequence
FIKVRQYDQILIEICGHK

SEQ ID NO: 70 - protein, 17 aa, synthetic construct
  source 1..17: note = OLP clade B consensus sequence
QILIEICGHKAIGTVLV

SEQ ID NO: 71 - protein, 18 aa, synthetic construct
  source 1..18: note = OLP clade B consensus sequence
LVGPTPVNIIGRNLLTQI

SEQ ID NO: 72 - protein, 17 aa, synthetic construct
  source 1..17: note = OLP clade B consensus sequence
LVEICTEMEKEGKISKI

SEQ ID NO: 73 - protein, 18 aa, synthetic construct
  source 1..18: note = OLP clade B consensus sequence
LDVGDAYFSVPLDKDFRK

SEQ ID NO: 74 - protein, 18 aa, synthetic construct
  source 1..18: note = OLP clade B consensus sequence
LRWGFTTPDKKHQKEPPF

SEQ ID NO: 75 - protein, 18 aa, synthetic construct
  source 1..18: note = OLP clade B consensus sequence
DKKHQKEPPFLWMGYELH

SEQ ID NO: 76 - protein, 15 aa, synthetic construct
  source 1..15: note = OLP clade B consensus sequence
EIQKQGQGQWTYQIY

SEQ ID NO: 77 - protein, 18 aa, synthetic construct
  source 1..18: note = OLP clade B consensus sequence
PPLVKLWYQLEKEPIVGA

SEQ ID NO: 78 - protein, 15 aa, synthetic construct
  source 1..15: note = OLP clade B consensus sequence
IHLALQDSGLEVNIV

SEQ ID NO: 79 - protein, 18 aa, synthetic construct
  source 1..18: note = OLP clade B consensus sequence
VYLAWVPAHKGIGGNEQV

SEQ ID NO: 80 - protein, 16 aa, synthetic construct
  source 1..16: note = OLP clade B consensus sequence
SAGIRKVLFLDGIDKA

SEQ ID NO: 81 - protein, 18 aa, synthetic construct
  source 1..18: note = OLP clade B consensus sequence
TKELQKQITKIQNFRVYY

SEQ ID NO: 82 - protein, 18 aa, synthetic construct
  source 1..18: note = OLP clade B consensus sequence
TKIQNFRVYYRDSRDPLW

SEQ ID NO: 83 - protein, 18 aa, synthetic construct
  source 1..18: note = OLP clade B consensus sequence
YYRDSRDPLWKGPAKLLW

SEQ ID NO: 84 - protein, 17 aa, synthetic construct
  source 1..17: note = OLP clade B consensus sequence
KIIRDYGKQMAGDDCVA

SEQ ID NO: 85 - protein, 18 aa, synthetic construct
  source 1..18: note = OLP clade B consensus sequence
GPQREPYNEWTLELLEEL

SEQ ID NO: 86 - protein, 18 aa, synthetic construct
  source 1..18: note = OLP clade B consensus sequence
DLNNNTNTTSSSGEKMEK

SEQ ID NO: 87 - protein, 18 aa, synthetic construct
  source 1..18: note = OLP clade B consensus sequence
IRDKVQKEYALFYKLDVV

SEQ ID NO: 88 - protein, 18 aa, synthetic construct
  source 1..18: note = OLP clade B consensus sequence
YRLISCNTSVITQACPKV

SEQ ID NO: 89 - protein, 18 aa, synthetic construct
  source 1..18: note = OLP clade B consensus sequence
SVITQACPKVSFEPIPIH

SEQ ID NO: 90 - protein, 16 aa, synthetic construct
  source 1..16: note = OLP clade B consensus sequence
TNVSTVQCTHGIRPVV

SEQ ID NO: 91 - protein, 18 aa, synthetic construct
  source 1..18: note = OLP clade B consensus sequence
VAPTKAKRRVVQREKRAV

SEQ ID NO: 92 - protein, 18 aa, synthetic construct
  source 1..18: note = OLP clade B consensus sequence
VIEVVQRACRAILHIPRR

SEQ ID NO: 93 - protein, 18 aa, synthetic construct
  source 1..18: note = OLP clade B consensus sequence
VKHHMYISGKAKGWFYRH

SEQ ID NO: 94 - protein, 18 aa, synthetic construct
  source 1..18: note = OLP clade B consensus sequence
GKAKGWFYRHHYESTHPR

SEQ ID NO: 95 - protein, 18 aa, synthetic construct
  source 1..18: note = OLP clade B consensus sequence
TKLTEDRWNKPQKTKGHR

SEQ ID NO: 96 - protein, 6 aa, synthetic construct
  source 1..6: note = Tag sequence
AHGHRP

SEQ ID NO: 97 - protein, 14 aa, synthetic construct
  source 1..14: note = Tag sequence
PIHDHDHPHLVIHS

SEQ ID NO: 98 - protein, 7 aa, synthetic construct
  source 1..7: note = Tag sequence
GMTCXXC

SEQ ID NO: 99 - protein, 530 aa, synthetic construct
  source 1..530: note = HIV immunogen comprising OLPs
EKIRLRPGGKKKYKLKHIVWASRELERFAVNPGLLETSEGCRQILGQLQPSLQTGSEELKSLYNTVATLYCVHQKIEVAAAKAFSPEVIPMFSALAAAGHQAAMQMLKEAAAIAPGQMREPRGSDIAGTTSTLQEQIGWMTNNPPIPVGEIYKRWIILGLNKIVRMYSPTSIAAAYVDRFYKTLRAEQAAACQGVGGPGHKARVLAAACTERQANFLGKIWPSHKGRPGNFLQSRAAAKMIGGIGGFIKVRQYDQILIEICGHKAIGTVLVGPTPVNIIGRNLLTQIGCTLNFAAALVEICTEMEKEGKISKIAAALRWGFTTPDKKHQKEPPFLWMGYELHPDKWTVQPIVLPEKDSWTVNDIQKLVGKLAAAILKEPVHGVYYDPSKDLIAEIQKQGQGQWTYQIYAAATKELQKQITKIQNFRVYYRDSRDPLWKGPAKLLWAAAKIIRDYGKQMAGDDCVAAAVKHHMYISKKAKGWFYRHHYESTHPRAAAVTKLTEDRWNKPQKTKGHRAAAWLEAQEEEEVGF

SEQ ID NO: 100 - DNA, 1587 bp, synthetic construct
  source 1..1587: mol_type = other DNA; note = polynucleotide encoding immunogen comprising HIV OLPs
gagaagatccggctgcggccaggcggaaagaagaagtacaagctgaagcacatcgtctgggcctcgagggagctggagcggttcgcggtgaacccgggacttctggagacgtcggaggggtgcaggcagatcctcggccagctgcagccctctctgcaaacggggtctgaggagctgaagagcctgtacaacacggtggcgaccctctactgcgtccaccagaagatcgaggtggcagcggccaaggcgttctcgccggaggtcatccccatgttctcggcgctggcagctgccggacaccaggccgcgatgcagatgctgaaggaggccgctgcgatcgcaccgggccagatgagggagccacgcggttccgacatcgcgggaaccacctcgacgctccaggagcagatcggatggatgacgaacaacccgccaatcccggtcggggagatctacaagcggtggatcatcctcgggctgaacaagatcgtccggatgtacagcccgacgtcgatcgctgcggcatacgttgaccggttctacaagaccctgagggccgagcaggcagcggcctgccagggggtcggtggaccagggcacaaggcccgagtgctcgcggccgcatgcacggagcggcaggcgaacttcctggggaagatctggccgtcgcacaagggccgaccgggaaacttcctccagtctcgcgcagcggctaagatgatcggaggcatcggaggcttcatcaaagtccgtcagtacgaccagatcctcatcgagatctgcgggcacaaggcgatcggaaccgtgctcgtcggcccaacgcccgtgaacatcatcggccgcaacctgttaacgcagatcggctgcaccctcaacttcgccgcactagtggagatctgcacggagatggagaaggagggcaagatatcgaagatcgcggcagctctgaggtggggcttcaccacgccggacaagaagcaccagaaggagccgccattcctgtggatgggatacgagctgcacccggacaagtggaccgtgcagcccatcgtcctgccggagaaggactcgtggacggtgaacgacatccagaagctcgtggggaagctggcggcagccatcctcaaggagcccgtccacggggtgtactacgacccctctaaggacctgatcgcggagatccagaagcaggggcagggtcagtggacctaccagatctacgcagcagcaaccaaggagctgcagaagcagatcacgaagatccagaacttccgcgtatactaccgcgactcgcgggaccccctgtggaagggccctgcgaagcttctctgggcagccgcgaagatcatccgggactacggcaagcagatggcgggcgacgactgcgtggccgcagcggtgaagcaccatatgtacatctcgaagaaggcgaagggctggttctacagacaccactacgagtccacccaccccagggcagctgcggtgacgaagctgacggaggaccggtggaacaagccccagaagacgaagggtcaccgggcggctgcatggctggaggctcaggaggaggaggaggtgggcttc

SEQ ID NO: 101 - protein, 15 aa, synthetic construct
  source 1..15: note = Gag p17
VEKIRLRPGGKKKYK

SEQ ID NO: 102 - protein, 15 aa, synthetic construct
  source 1..15: note = Gag p17
RLRPGGKKKYKLKHI

SEQ ID NO: 103 - protein, 14 aa, synthetic construct
  source 1..14: note = Gag p17
GGKKKYKLKHIVWA

SEQ ID NO: 104 - protein, 15 aa, synthetic construct
  source 1..15: note = Gag p17
KKYKLKHIVWASREL

SEQ ID NO: 105 - protein, 15 aa, synthetic construct
  source 1..15: note = Gag p17
LKHIVWASRELERFA

SEQ ID NO: 106 - protein, 12 aa, synthetic construct
  source 1..12: note = Gag p17
VWASRELERFAV

SEQ ID NO: 107 - protein, 15 aa, synthetic construct
  source 1..15: note = Gag p17
WASRELERFAVNPGL

SEQ ID NO: 108 - protein, 12 aa, synthetic construct
  source 1..12: note = Gag p17
ELERFAVNPGLL

SEQ ID NO: 109 - protein, 15 aa, synthetic construct
  source 1..15: note = Gag p17
LERFAVNPGLLETSE

SEQ ID NO: 110 - protein, 14 aa, synthetic construct
  source 1..14: note = Gag p17
AVNPGLLETSEGCR

SEQ ID NO: 111 - protein, 14 aa, synthetic construct
  source 1..14: note = Gag p17
PGLLETSEGCRQIL

SEQ ID NO: 112 - protein, 14 aa, synthetic construct
  source 1..14: note = Gag p17
LETSEGCRQILGQL

SEQ ID NO: 113 - protein, 15 aa, synthetic construct
  source 1..15: note = Gag p17
SEGCRQILGQLQPSL

SEQ ID NO: 114 - protein, 15 aa, synthetic construct
  source 1..15: note = Gag p17
RQILGQLQPSLQTGS

SEQ ID NO: 115 - protein, 15 aa, synthetic construct
  source 1..15: note = Gag p17
GQLQPSLQTGSEELK

SEQ ID NO: 116 - protein, 14 aa, synthetic construct
  source 1..14: note = Gag p17
PSLQTGSEELKSLY

SEQ ID NO: 117 - protein, 15 aa, synthetic construct
  source 1..15: note = Gag p17
QTGSEELKSLYNTVA

SEQ ID NO: 118 - protein, 14 aa, synthetic construct
  source 1..14: note = Gag p17
EELKSLYNTVATLY

SEQ ID NO: 119 - protein, 14 aa, synthetic construct
  source 1..14: note = Gag p17
KSLYNTVATLYCVH

SEQ ID NO: 120 - protein, 14 aa, synthetic construct
  source 1..14: note = Gag p17
YNTVATLYCVHQKI

SEQ ID NO: 121 - protein, 15 aa, synthetic construct
  source 1..15: note = Gag p17
VATLYCVHQKIEVAA

SEQ ID NO: 122 - protein, 15 aa, synthetic construct
  source 1..15: note = Gag p24
KAFSPEVIPMFSALA

SEQ ID NO: 123 - protein, 15 aa, synthetic construct
  source 1..15: note = Gag p24
AGHQAAMQMLKEAAA

SEQ ID NO: 124 - protein, 15 aa, synthetic construct
  source 1..15: note = Gag p24
AIAPGQMREPRGSDI

SEQ ID NO: 125 - protein, 12 aa, synthetic construct
  source 1..12: note = Gag p24
GQMREPRGSDIA

SEQ ID NO: 126 - protein, 17 aa, synthetic construct
  source 1..17: note = Gag p24
QMREPRGSDIAGTTSTL

SEQ ID NO: 127 - protein, 15 aa, synthetic construct
  source 1..15: note = Gag p24
GSDIAGTTSTLQEQI

SEQ ID NO: 128 - protein, 14 aa, synthetic construct
  source 1..14: note = Gag p24
AGTTSTLQEQIGWM

SEQ ID NO: 129 - protein, 17 aa, synthetic construct
  source 1..17: note = Gag p24
TSTLQEQIGWMTNNPPI

SEQ ID NO: 130 - protein, 13 aa, synthetic construct
  source 1..13: note = Gag p24
QIGWMTNNPPIPV

SEQ ID NO: 131 - protein, 15 aa, synthetic construct
  source 1..15: note = Gag p24
GWMTNNPPIPVGEIY

SEQ ID NO: 132 - protein, 15 aa, synthetic construct
  source 1..15: note = Gag p24
NNPPIPVGEIYKRWI

SEQ ID NO: 133 - protein, 15 aa, synthetic construct
  source 1..15: note = Gag p24
IPVGEIYKRWIILGL

SEQ ID NO: 134 - protein, 15 aa, synthetic construct
  source 1..15: note = Gag p24
EIYKRWIILGLNKIV

SEQ ID NO: 135 - protein, 14 aa, synthetic construct
  source 1..14: note = Gag p24
RWIILGLNKIVRMY

SEQ ID NO: 136 - protein, 16 aa, synthetic construct
  source 1..16: note = Gag p24
ILGLNKIVRMYSPTSI

SEQ ID NO: 137 - protein, 15 aa, synthetic construct
  source 1..15: note = Gag p24
YVDRFYKTLRAEQAA

SEQ ID NO: 138 - protein, 15 aa, synthetic construct
  source 1..15: note = Gag p24
AACQGVGGPGHKARV

SEQ ID NO: 139 - protein, 15 aa, synthetic construct
  source 1..15: note = Gag p24
GVGGPGHKARVLAAA

SEQ ID NO: 140 - protein, 15 aa, synthetic construct
  source 1..15: note = Gag p2p7p1p6
AAACTERQANFLGKI

SEQ ID NO: 141 - protein, 15 aa, synthetic construct
  source 1..15: note = Gag p2p7p1p6
TERQANFLGKIWPSH

SEQ ID NO: 142 - protein, 14 aa, synthetic construct
  source 1..14: note = Gag p2p7p1p6
ANFLGKIWPSHKGR

SEQ ID NO: 143 - protein, 15 aa, synthetic construct
  source 1..15: note = Gag p2p7p1p6
LGKIWPSHKGRPGNF

SEQ ID NO: 144 - protein, 15 aa, synthetic construct
  source 1..15: note = Gag p2p7p1p6
WPSHKGRPGNFLQSR

SEQ ID NO: 145 - protein, 14 aa, synthetic construct
  source 1..14: note = Pol Prot
AKMIGGIGGFIKVR

SEQ ID NO: 146 - protein, 13 aa, synthetic construct
  source 1..13: note = Pol Prot
IGGIGGFIKVRQY

SEQ ID NO: 147 - protein, 15 aa, synthetic construct
  source 1..15: note = Pol Prot
GIGGFIKVRQYDQIL

SEQ ID NO: 148 - protein, 14 aa, synthetic construct
  source 1..14: note = Pol Prot
FIKVRQYDQILIEI

SEQ ID NO: 149 - protein, 15 aa, synthetic construct
  source 1..15: note = Pol Prot
VRQYDQILIEICGHK

SEQ ID NO: 150 - protein, 13 aa, synthetic construct
  source 1..13: note = Pol Prot
DQILIEICGHKAI

SEQ ID NO: 151 - protein, 15 aa, synthetic construct
  source 1..15: note = Pol Prot
ILIEICGHKAIGTVL

SEQ ID NO: 152 - protein, 12 aa, synthetic construct
  source 1..12: note = Pol Prot
ICGHKAIGTVLV

SEQ ID NO: 153 - protein, 16 aa, synthetic construct
  source 1..16: note = Pol Prot
CGHKAIGTVLVGPTPV

SEQ ID NO: 154 - protein, 14 aa, synthetic construct
  source 1..14: note = Pol Prot
IGTVLVGPTPVNII

SEQ ID NO: 155 - protein, 15 aa, synthetic construct
  source 1..15: note = Pol Prot
VLVGPTPVNIIGRNL

SEQ ID NO: 156 - protein, 15 aa, synthetic construct
  source 1..15: note = Pol Prot
PTPVNIIGRNLLTQI

SEQ ID NO: 157 - protein, 15 aa, synthetic construct
  source 1..15: note = Pol Prot
NIIGRNLLTQIGCTL

SEQ ID NO: 158 - protein, 15 aa, synthetic construct
  source 1..15: note = Pol Prot
RNLLTQIGCTLNFAA

SEQ ID NO: 159 - protein, 15 aa, synthetic construct
  source 1..15: note = Pol RT
ALVEICTEMEKEGKI

SEQ ID NO: 160 - protein, 15 aa, synthetic construct
  source 1..15: note = Pol RT
ICTEMEKEGKISKIA

SEQ ID NO: 161 - protein, 15 aa, synthetic construct
  source 1..15: note = Pol RT
ALRWGFTTPDKKHQK

SEQ ID NO: 162 - protein, 15 aa, synthetic construct
  source 1..15: note = Pol RT
GFTTPDKKHQKEPPF

SEQ ID NO: 163 - protein, 14 aa, synthetic construct
  source 1..14: note = Pol RT
PDKKHQKEPPFLWM

SEQ ID NO: 164 - protein, 15 aa, synthetic construct
  source 1..15: note = Pol RT
KHQKEPPFLWMGYEL

SEQ ID NO: 165 - protein, 15 aa, synthetic construct
  source 1..15: note = Pol RT
EPPFLWMGYELHPDK

SEQ ID NO: 166 - protein, 14 aa, synthetic construct
  source 1..14: note = Pol RT
LWMGYELHPDKWTV

SEQ ID NO: 167 - protein, 15 aa, synthetic construct
  source 1..15: note = Pol RT
GYELHPDKWTVQPIV

SEQ ID NO: 168 - protein, 15 aa, synthetic construct
  source 1..15: note = Pol RT
HPDKWTVQPIVLPEK

SEQ ID NO: 169 - protein, 14 aa, synthetic construct
  source 1..14: note = Pol RT
WTVQPIVLPEKDSW

SEQ ID NO: 170 - protein, 13 aa, synthetic construct
  source 1..13: note = Pol RT
QPIVLPEKDSWTV

SEQ ID NO: 171 - protein, 14 aa, synthetic construct
  source 1..14: note = Pol RT
IVLPEKDSWTVNDI

SEQ ID NO: 172 - protein, 15 aa, synthetic construct
  source 1..15: note = Pol RT
PEKDSWTVNDIQKLV

SEQ ID NO: 173 - protein, 15 aa, synthetic construct
  source 1..15: note = Pol RT
SWTVNDIQKLVGKLA

SEQ ID NO: 174 - protein, 12 aa, synthetic construct
  source 1..12: note = Pol RT
AILKEPVHGVYY

SEQ ID NO: 175 - protein, 15 aa, synthetic construct
  source 1..15: note = Pol RT
ILKEPVHGVYYDPSK

SEQ ID NO: 176 - protein, 15 aa, synthetic construct
  source 1..15: note = Pol RT
PVHGVYYDPSKDLIA

SEQ ID NO: 177 - protein, 15 aa, synthetic construct
  source 1..15: note = Pol RT
VYYDPSKDLIAEIQK

SEQ ID NO: 178 - protein, 17 aa, synthetic construct
  source 1..17: note = Pol RT
PSKDLIAEIQKQGQGQW

SEQ ID NO: 179 - protein, 15 aa, synthetic construct
  source 1..15: note = Pol RT
AEIQKQGQGQWTYQI

SEQ ID NO: 180 - protein, 15 aa, synthetic construct
  source 1..15: note = Pol RT
KQGQGQWTYQIYAAA

SEQ ID NO: 181 - protein, 15 aa, synthetic construct
  source 1..15: note = Pol Int
TKELQKQITKIQNFR

SEQ ID NO: 182 - protein, 15 aa, synthetic construct
  source 1..15: note = Pol Int
QKQITKIQNFRVYYR

SEQ ID NO: 183 - protein, 14 aa, synthetic construct
  source 1..14: note = Pol Int
TKIQNFRVYYRDSR

SEQ ID NO: 184 - protein, 15 aa, synthetic construct
  source 1..15: note = Pol Int
QNFRVYYRDSRDPLW

SEQ ID NO: 185 - protein, 15 aa, synthetic construct
  source 1..15: note = Pol Int
VYYRDSRDPLWKGPA

SEQ ID NO: 186 - protein, 15 aa, synthetic construct
  source 1..15: note = Pol Int
DSRDPLWKGPAKLLW

SEQ ID NO: 187 - protein, 16 aa, synthetic construct
  source 1..16: note = Pol Int
KIIRDYGKQMAGDDCV

SEQ ID NO: 188 - protein, 14 aa, synthetic construct
  source 1..14: note = Vif Vif
AAVKHHMYISKKAK

SEQ ID NO: 189 - protein, 15 aa, synthetic construct
  source 1..15: note = Vif Vif
KHHMYISKKAKGWFY

SEQ ID NO: 190 - protein, 15 aa, synthetic construct
  source 1..15: note = Vif Vif
YISKKAKGWFYRHHY

SEQ ID NO: 191 - protein, 15 aa, synthetic construct
  source 1..15: note = Vif Vif
KAKGWFYRHHYESTH

SEQ ID NO: 192 - protein, 15 aa, synthetic construct
  source 1..15: note = Vif Vif
WFYRHHYESTHPRAA

SEQ ID NO: 193 - protein, 14 aa, synthetic construct
  source 1..14: note = Vif Vif
VTKLTEDRWNKPQK

SEQ ID NO: 194 - protein, 15 aa, synthetic construct
  source 1..15: note = Vif Vif
LTEDRWNKPQKTKGH

SEQ ID NO: 195 - protein, 15 aa, synthetic construct
  source 1..15: note = Vif Vif
RWNKPQKTKGHRAAA

SEQ ID NO: 196 - protein, 13 aa, synthetic construct
  source 1..13: note = Nef Nef
AWLEAQEEEEVGF

SEQ ID NO: 197 - protein, 15 aa, synthetic construct
  source 1..15: note = Gag-Linker p24
HQKIEVAAAKAFSPE

SEQ ID NO: 198 - protein, 15 aa, synthetic construct
  source 1..15: note = Gag-Linker p24
PMFSALAAAGHQAAM

SEQ ID NO: 199 - protein, 15 aa, synthetic construct
  source 1..15: note = Gag-Linker p24
MQMLKEAAAIAPGQM

SEQ ID NO: 200 - protein, 15 aa, synthetic construct
  source 1..15: note = Gag-Linker p24
YSPTSIAAAYVDRFT

SEQ ID NO: 201 - protein, 13 aa, synthetic construct
  source 1..13: note = Gag-Linker p24
LRAEQAAACQGVG

SEQ ID NO: 202 - protein, 15 aa, synthetic construct
  source 1..15: note = Gag-Linker p24
HKARVLAAACTERQA

SEQ ID NO: 203 - protein, 15 aa, synthetic construct
  source 1..15: note = Pol-Linker Prot
NFLQSRAAAKMIGGI

SEQ ID NO: 204 - protein, 14 aa, synthetic construct
  source 1..14: note = Pol-Linker RT
GCTLNFAALVEICT

SEQ ID NO: 205 - protein, 15 aa, synthetic construct
  source 1..15: note = Pol-Linker RT
GKISKIAAALRWGFT

SEQ ID NO: 206 - protein, 15 aa, synthetic construct
  source 1..15: note = Pol-Linker RT
KLVGKLAAAILKEPV

SEQ ID NO: 207 - protein, 15 aa, synthetic construct
  source 1..15: note = Pol-Linker Int
WTYQIYAAATKELQK

SEQ ID NO: 208 - protein, 15 aa, synthetic construct
  source 1..15: note = Pol-Linker Int
PAKLLWAAAKIIRDY

SEQ ID NO: 209 - protein, 15 aa, synthetic construct
  source 1..15: note = Pol-Linker Int
AGDDCVAAAVKHHMY

SEQ ID NO: 210 - protein, 15 aa, synthetic construct
  source 1..15: note = Vif-Linker Vif
ESTHPRAAAVTKLTE

SEQ ID NO: 211 - protein, 15 aa, synthetic construct
  source 1..15: note = Nef-Linker Nef
KTKGHRAAAWLEAQE

(19) United States
(12) Patent Application Publication
(10) Pub. No.: US 2026/0063627 A1
(43) Pub. Date: Mar. 5, 2026

(54) METHOD OF TREATMENT OF HIV INFECTION WITH VACCINE

(71) Applicant: Gilead Sciences, Inc., Foster City, CA (US)

(72) Inventors: Christian BRANDER, Tiana (ES); Beatriz MOTHE, Tiana (ES); Ian MCGOWAN, Barcelona (ES); Yovaninna ALARCÓN-SOTO, Barcelona (ES)

(21) Appl. No.: 19/065,036

(22) Filed: Feb. 27, 2025

Related U.S. Application Data
(63) Continuation of application No. 18/778,595, filed on Jul. 19, 2024, now abandoned.
(60) Provisional application No. 63/514,760, filed on Jul. 20, 2023.

(51) Int. Cl. G01N 33/543; A61K 31/7088; A61K 38/16; A61K 39/21; A61K 45/06; A61P 31/18; C12N 15/861; G01N 33/50; G01N 33/68

(52) U.S. Cl. CPC G01N 33/54306; A61K 31/7088; A61K 38/162; A61K 39/21; A61K 45/06; A61P 31/18; C12N 15/861; G01N 33/5005; G01N 33/6893; G01N 2333/57; G01N 2800/26; G01N 2800/52

(57) ABSTRACT

The present disclosure relates to methods for determining the magnitude of a subject's immune response against a HIVACAT T-cell immunogen (HTI or “HTI immunogen”) and whether the subject can avoid antiretroviral therapy (ART). These methods are helpful for treating human immunodeficiency virus (HIV) and/or deciding whether to administer, continue or stop antiretroviral therapy in a subject. The present disclosure also relates to antigens, compositions, and kits related to such methods.

### CROSS-REFERENCE TO RELATED APPLICATIONS

This application is a continuation of U.S. application Ser. No. 18/778,595, filed Jul. 19, 2024, which claims priority to and benefit of U.S. Provisional Application No. 63/514,760, filed Jul. 20, 2023, both of which are hereby incorporated by reference in their entireties.

### REFERENCE TO SEQUENCE LISTING SUBMITTED ELECTRONICALLY

The content of the electronically submitted sequence listing (Name: 3834_0120001_Seqlisting_ST26; Size: 228,333 bytes; and Date of Creation: Jul. 17, 2024) is herein incorporated by reference in its entirety.

### FIELD OF THE DISCLOSURE

The present disclosure relates to methods for determining the magnitude of a subject's immune response to a HIVACAT T-cell immunogen (HTI or “HTI immunogen”) and whether the subject can avoid or interrupt antiretroviral therapy (ART). These methods are helpful for treating human immunodeficiency virus (HIV) and/or deciding whether to administer, continue, or stop antiretroviral therapy in a subject. The present disclosure also relates to antigens, compositions, and kits related to such methods.

### BACKGROUND

Human immunodeficiency virus (HIV) was first identified and described as the cause of acquired immunodeficiency syndrome (AIDS) four decades ago. Since then, nearly 80 million people have become infected with HIV, from which more than 36 million people have died from AIDS-related illnesses. Nowadays, almost 40 million people globally are living with HIV (PLWH), with 1.5 million people newly infected in 2022. (Joint United Nations Programme on HIV/AIDS, “UNAIDS Data 2022,” available at www.unaids.org/en/resources/documents/2023/2022_unaids_data) The development of effective antiretroviral therapies (ART) represented a turning point in the control of HIV transmission, as well as in improving the quality of life of people living with HIV. However, only about 27 million people could access ART in 2020, meaning that more than 10 million people were at potential risk of suffering from other infections, as well as acting as vectors of HIV transmission.

Under current standards of care, patients are treated with a combination of antiretroviral therapies including two or more highly effective ART using different mechanisms of action (combined ARTi following HIV diagnosis. This raises a huge challenge not only for health systems, but also for PLWH, as ART is a lifetime of daily medication that can extend in some cases beyond 60 years. This has several implications including, but not limited to, 1) the emergence of long-term adverse effects of medications; 2) adherence fatigue resulting in development of antiretroviral resistance, risk to infect sexual partners due to an inadequately suppressed virus, associated poor health outcomes and increased resource utilization, among others; 3) costs related to routine clinical monitoring and investigations; and 4) challenging high costs to patients or health systems associated to long term chronic medication supply (prescription fees and patient out-of-pocket costs, wholesalers and pharmacy margins).

In this context, prevention of HIV infection as well as therapeutic treatment for HIV has become a challenging major unmet need for society.

Many strategies to prevent and cure HIV have already been assessed. These include the use of chemical HIV replication inhibitors, latency reversal agents, and cellular immunity therapies elicited by vaccines, soluble native-like HIV Env trimers (SOSIPs) and/or broadly neutralizing antibodies (bNAbs). However, none of these strategies have per se succeeded in the complete control and eradication of HIV from infected people. Thus, it is most likely that a combination of different approaches might be the preferred scheme leading to a final effective therapy to cure HIV.

T-cell vaccines are an essential component of a cure-eradication strategy. In addition to the hugely efficient development of ART and despite decades of effort, prophylaxis of HIV-1 transmission has been less successful as the development of an efficient HIV-1 vaccine has not been achieved yet. The high protein sequence variability among HIV strains together with effective features of the HIV-1 proteins that decreases the neutralizing potency of antibodies are identified as powerful weapons that allow the virus to avoid the immune response. Therefore, the development of potent immunogens able to effectively induce antiviral-specific T-cell responses is crucial.

HIVACAT T-cell immunogen (HTI) is a fusion protein composed of 16 segments of HIV-1, each having from about 11 to about 78 amino acids in length, and encoding critical targets of viral proteins Gag (45%), Pol (44%), Vif (8%) and Nef (3%). (See U.S. Pat. No. 9,988,425, which is incorporated by reference in its entirety.) These epitopes were identified as viral targets associated with relative HIV-1 viral control in a comprehensive screening of large cohorts of clade B and C HIV-1-infected participants. This design is distinct from previous designs that were based on full protein sequences, very short and conserved segments of the virus, conserved CD8 T-cell epitopes or other aspects not based on relevant clinical data. The design of HTI and its efficacy to control HIV replication after cART interruption have been described. (Bailon L et al., Safety, immunogenicity and effect on viral rebound of HTI vaccines in early treated HIV-1 infection: a randomized, placebo-controlled phase 1 trial; Nat Med. 2022 December; 28 (12): 2611-2621. doi: 10.1038/s41591-022-02060-2.)

As one can expect some degree of variability from subject to subject in the immune response upon vaccination with HTI, as well as different degrees of efficacy on HIV control after vaccination, in the clinical practice a clinician needs a method to complete HTI treatment and interrupt the current antiretroviral vaccination. This method should include an objective measurement that predicts which HTI-vaccinated patients can sustain long-term virus control upon antiretroviral interruption. Measuring the magnitude of an immune response against HTI in subjects vaccinated with HTI allows the clinician or treating person to safely interrupt the antiretroviral treatment and to successfully predict those who will have a long term control of the HIV viral load.

In addition, the magnitude of immune response against HTI can be used in multiple settings not including a prior HTI vaccination, to determine the probability of HIV control in the decision to administer or not administer antiretroviral medication. For example, measuring the magnitude of the immune response against HTI can be used to decide if a patient should start antiretrovirals or not for the first time, or after a pause, or it can be used to decide if a patient can interrupt antiretrovirals after a vaccination regime with another vaccine that does not include HTI.

Moreover, the magnitude of the immune response against HTI can be used in the preventive setting, i.e., after vaccination with HTI (or with any other HIV vaccine not including HTI) of the general population not infected with HIV. In this case, the magnitude of the immune response against HTI can be used as surrogate for vaccine efficacy and for level of protection, for example, indicating the need of additional vaccine boosters.

### BRIEF SUMMARY

The present disclosure provides a method of treating a human immunodeficiency (HIV) infection in a human subject undergoing treatment with an antiretroviral therapy, comprising (a) administering a HIVACAT T-cell immunogen (HTI or “HTI immunogen”) to the subject; (b) evaluating the magnitude of the subject's immune response to the HTI immunogen; and (c) stopping treatment with the antiretroviral therapy if the subject has an elevated immune response to the HTI immunogen.

The present disclosure also provides a method of treating an HIV infection in a human subject undergoing treatment with an antiretroviral therapy, comprising (a) administering an HTI immunogen to the subject; (b) evaluating the magnitude of the subject's immune response to the HTI immunogen; and (c) continuing treatment with the antiretroviral therapy if the subject does not have an elevated immune response to the HTI immunogen.

The present disclosure also provides a method of treating an HIV infection in a human subject that is not undergoing treatment with an antiretroviral therapy, comprising (a) administering an HTI immunogen to the subject; (b) evaluating the magnitude of the subject's immune response to the HTI immunogen; and (c) initiating antiretroviral therapy if the subject does not have an elevated immune response to the HTI immunogen.

The present disclosure also provides a method for identifying a human subject infected with HIV that can stop treatment with an antiretroviral therapy, comprising (a) administering an HTI immunogen to the subject; (b) evaluating the magnitude of the subject's immune response to the HTI immunogen; and (c) determining that the subject can stop treatment with the antiretroviral therapy if the subject has an elevated immune response to the HTI immunogen.

The present disclosure also provides a method for identifying a human subject infected with HIV that can continue treatment with an antiretroviral therapy, comprising (a) administering an HTI immunogen to the subject; (b) evaluating the magnitude of the subject's immune response to the HTI immunogen; and (c) identifying that the subject can continue treatment with the antiretroviral therapy if the subject does not have an elevated immune response to the HTI immunogen.

The present disclosure also provides a method of identifying a human subject infected with HIV who can start treatment with an antiretroviral therapy, comprising (a) administering an HTI immunogen to the subject; (b) evaluating the magnitude of the subject's immune response to the HTI immunogen; and (c) determining that the subject can start treatment with the antiretroviral therapy if the subject does not have an elevated immune response to the HTI immunogen.

The present disclosure also provides a method of identifying a human subject infected with HIV that can stop treatment with an antiretroviral therapy, comprising (a) administering an HIV vaccine not including HTI to the subject; (b) evaluating the magnitude of the subject's immune response to an HTI immunogen; and (c) determining that the subject can stop treatment with the antiretroviral therapy if the subject has an elevated immune response to the HTI immunogen.

The present disclosure also provides a method of identifying a human subject that has been successfully vaccinated against HIV in the preventive setting, comprising (a) administering an HTI immunogen to the subject; (b) evaluating the magnitude of the subject's immune response to the HTI immunogen; and (c) determining that the subject has mounted a protective response against HIV infection.

The present disclosure also provides a method of identifying a human subject that has been successfully vaccinated against HIV in the preventive setting, comprising (a) administering an HIV vaccine not including HTI to the subject; (b) evaluating the magnitude of the subject's immune response to an HTI immunogen; and (c) determining that the subject has mounted a protective response against HIV infection.

The present disclosure also provides a method for evaluating the magnitude of an immune response in a subject, comprising (a) administering an HTI immunogen to the subject; and (b) evaluating magnitude of the subject's immune response to the HTI immunogen.

The present disclosure also provides an HTI immunogen for evaluating magnitude of an immune response in a biological sample.

The present disclosure also provides an HTI immunogen for identifying a human subject infected with HIV that can stop treatment with an antiretroviral therapy.

The present disclosure also provides an HTI immunogen for identifying a human subject infected with HIV that can continue treatment with an antiretroviral therapy.

The present disclosure also provides an HTI immunogen for identifying a human subject infected with HIV that can start treatment with an antiretroviral therapy. The present disclosure also provides a composition or kit comprising such HTI immunogens.

### BRIEF DESCRIPTION OF THE DRAWINGS

Some aspects of the disclosure are herein described, by way of example only, with reference to the accompanying drawings. With specific reference now to the drawings in detail, it is stressed that the particulars shown are by way of example and for purposes of illustrative discussion of aspects of the invention.

FIG. 1 is a diagram showing the phases of a clinical study and investigational medicinal product administration schedule described in Example 1. D=administration of HIVACAT T-cell immunogen (HTI) in a DNA vector. M=administration of HTI in a Modified Vaccinia Ankara (MVA) vector. C=administration of HTI in a ChAdOx adenovirus vector. P=administration of placebo. ATI=analytical treatment interruption. SCR=screening.

FIG. 2 shows the median magnitude of total HTI-specific T-cells at ATI start from N=26 data points.

FIG. 3 shows the correlation between magnitude of total HTI-specific T-cell response at ATI start and time off antiretroviral therapy (ART). The dashed line depicts the threshold value (median of magnitude total-HTI of specific T-cells) set for the stratification of participants.

FIG. 4 shows survival curves between participants from active and placebo arms by magnitude of HTI-specific responses at ATI start with stratification corresponding to High-HTI as > median of magnitude total HTI-specific T-cells and low-HTI as < median magnitude Total HTI-specific T-cells.

### DETAILED DESCRIPTION

The present disclosure relates to methods for determining in a human subject the magnitude of an immune response against a HIVACAT T-cell immunogen (HTI or “HTI immunogen”) and whether the subject can avoid antiretroviral therapy (ART). These methods are helpful for treating human immunodeficiency virus (HIV) and/or deciding whether to administer, continue, or stop antiretroviral therapy in a subject. The present disclosure also relates to antigens, compositions, and kits related to such methods.

### I. General Definitions

Unless defined otherwise, all technical and scientific terms used herein have the same meaning as commonly understood by one of ordinary skill in the art to which this disclosure belongs. In case of conflict, the present application, including the definitions, will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

By “sequence identity” or “sequence similarity” is meant that the identity or similarity between two or more amino acid sequences, or two or more nucleotide sequences, expressed in terms of the identity or similarity between the sequences. Sequence identity can be measured in terms of “percentage (%) identity,” wherein the higher the percentage, the more identity shared between the sequences. Sequence similarity can be measured in terms of percentage similarity (which takes into account conservative amino acid substitutions); the higher the percentage, the more similarity shared between the sequences. Homologs or orthologs of nucleic acid or amino acid sequences possess a relatively high degree of sequence identity/similarity when aligned using standard methods. Sequence identity can be measured using sequence analysis software on the default setting (e.g., Sequence Analysis Software Package of the Genetics Computer Group, University of Wisconsin Biotechnology Center, 17 10 University Avenue, Madison, WI 53705). Such software can match similar sequences by assigning degrees of homology to various substitutions, deletions, and other modifications.

“Percentage of sequence identity” is determined by comparing two optimally aligned sequences over a comparison window, wherein the portion of the nucleic acid or polypeptide sequence in the comparison window can have additions or deletions (i.e., gaps) as compared to the reference sequence (which does not have additions or deletions) for optimal alignment of the two sequences. In some cases, the percentage can be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

“Identical” or percent “identity” in the context of two or more nucleic acid or polypeptide sequences, refer to two or more sequences or subsequences that are the same or have a specified percentage of amino acid residues or nucleotides that are the same (e.g., 60%, 65%, 70%, 75%, 80%, 85%, 90%, 95%, 98%, or 99% identity over a specified region, e.g., the entire polypeptide sequences or individual domains of the polypeptides), when compared and aligned for maximum correspondence over a comparison window or designated region as measured using one of the following sequence comparison algorithms or by manual alignment and visual inspection. Such sequences are then said to be “substantially identical.” This definition also refers to the complement of a test sequence.

“Nucleic acid,” as used herein, refers to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. A nucleic acid can comprise modified nucleotides, such as methylated nucleotides and their analogs.

“Treatment” or “treat” or “treating,” as used herein, refer to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

As used herein, “administering” is meant a method of giving a dosage of an HTI immunogen or composition provided herein to a subject. Administration can be, for example, intramuscularly, intravenously, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subcutaneously, subconjunctival, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, by gavage, in cremes, or in lipid compositions. The preferred method of administration can vary depending on various factors (e.g., the components of the composition being administered and the seventy of the condition being treated).

The term “virus,” as used herein, is defined as an infectious agent that is unable to grow or reproduce outside a host cell and that infects mammals (e.g., humans) or birds. Human immunodeficiency virus (HIV) is a representative virus.

By “human immunodeficiency virus” or “HIV” is meant a virus of the genus Lentivirinae, part of the family of Retroviridae, and includes, but is not limited to, HIV type 1 (HIV-1) and HIV type 2 (HIV-2), two species of HIV that infect humans.

### II. Methods, Antigens, Compositions and Kits

The present disclosure relates to methods for determining the magnitude of a subject's immune response against a HIVACAT T-cell immunogen (HTI or “HTI immunogen”) in a human subject infected with HIV and whether the subject can avoid antiretroviral therapy (ART). These methods are helpful for treating human immunodeficiency virus (HIV) and/or deciding whether to administer, continue, or stop antiretroviral therapy in a subject. The present disclosure also relates to antigens, compositions, and kits related to such methods.

More specifically, in some aspects, the present disclosure relates to a method of treating an HIV infection in a human subject undergoing treatment with an antiretroviral therapy, comprising (a) administering an HTI immunogen to the subject; (b) evaluating the magnitude of the subject's immune response to the HTI immunogen; and (c) stopping treatment with the antiretroviral therapy if the subject has an elevated immune response to the HTI immunogen.

In some aspects, the present disclosure relates to a method of treating an HIV infection in a human subject undergoing treatment with an antiretroviral therapy, comprising (a) administering an HTI immunogen to the subject; (b) evaluating the magnitude of the subject's immune response to the HTI immunogen; and (c) continuing treatment with the antiretroviral therapy if the subject does not have an elevated immune response to the HTI immunogen.

In some aspects, the present disclosure relates to a method of treating an HIV infection in a human subject, comprising (a) administering an HTI immunogen to the subject; (b) evaluating the magnitude of the subject's immune response to the HTI immunogen; and (c) starting treatment with an antiretroviral therapy if the subject does not have an elevated immune response to the HTI immunogen.

In some aspects, the present disclosure relates to a method for identifying a human subject infected with HIV who can stop treatment with an antiretroviral therapy, comprising (a) administering an HTI immunogen to the subject; (b) evaluating the magnitude of the subject's immune response to the HTI immunogen; and (c) identifying that the subject can stop treatment with the antiretroviral therapy if the subject has an elevated immune response to the HTI immunogen.

In some aspects, the present disclosure relates to a method for identifying a human subject infected with HIV who can continue treatment with an antiretroviral therapy, comprising (a) administering an HTI immunogen to the subject; (b) evaluating the magnitude of the subject's immune response to the HTI immunogen; and (c) identifying that the subject can continue treatment with the antiretroviral therapy if the subject does not have an elevated immune response to the HTI immunogen.

In some aspects, the present disclosure relates to a method of identifying a human subject infected with HIV who can start treatment with an antiretroviral therapy, comprising (a) administering an HTI immunogen to the subject; (b) evaluating the magnitude of the subject's immune response to the HTI immunogen; and (c) identifying that the subject can start treatment with the antiretroviral therapy if the subject does not have an elevated immune response to the HTI immunogen.

In some aspects, the present disclosure relates to a method for evaluating the magnitude of an immune response in a subject, comprising (a) administering an HTI immunogen to the subject; and (b) evaluating magnitude of the subject's immune response to the HTI immunogen.

In some aspects, the present disclosure relates to a method of treatment of HIV infection in a subject with an HTI immunogen and assessment of efficacy of the treatment.

In some aspects, the method comprises (a) vaccination of the subject with the HTI immunogen; (b) obtaining biological samples of PBMC of the vaccinated subject at specific timepoints before, during and after the vaccination therapy; (c) assessing the magnitude of the immune response to an HTI antigen (biomarker); and determining whether or not the biomarker is above or below a threshold value; and (d) determining if the subject with HIV is eligible to complete the treatment.

In some aspects, the present disclosure relates to a method of identifying a human subject infected with HIV that can stop treatment with an antiretroviral therapy, comprising (a) administering an HIV vaccine not including HTI to the subject; (b) evaluating the magnitude of the subject's immune response to an HTI immunogen; and (c) determining that the subject can stop treatment with the antiretroviral therapy if the subject has an elevated immune response to the HTI immunogen.

In some aspects, the present disclosure relates to a method of identifying a human subject that has been successfully vaccinated against HIV in the preventive setting, comprising (a) administering an HTI immunogen to the subject; (b) evaluating the magnitude of the subject's immune response to the HTI immunogen; and (c) determining that the subject has mounted a protective response against HIV infection.

In some aspects, the present disclosure relates to a method of identifying a human subject that has been successfully vaccinated against HIV in the preventive setting, comprising (a) administering an HIV vaccine not including HTI to the subject; (b) evaluating the magnitude of the subject's immune response to an HTI immunogen; and (c) determining that the subject has mounted a protective response against HIV infection.

As used herein, the term “HTI” refers to “HIVACAT T-cell immunogen.” (U.S. Pat. No. 9,988,425, for example.)

As used herein, the term “HTI immunogen” refers to an immunogenic fusion protein constructed from 16 segments of human immunodeficiency virus type 1 (HIV-1), each having from about 11 to about 78 amino acids in length, and encoding critical epitopes of the viral proteins Gag (45%), Pol (44%), Vif (8%) and Nef (3%) as described, for example, in Mothe, B. et al., J. Transl. Med. 13:60 (2015) and U.S. Pat. No. 9,988,425 (each of which is incorporated herein by reference in its entirety).

In some aspects, the HTI immunogen is a polypeptide comprising (i) a sequence having at least 90%, at least 91%, at least 92%, at least 93%, at least 94%, at least 95%, at least 96%, at least 97%, at least 98%, or at least 99% identity to SEQ ID NO:1; (ii) a sequence having at least 90% identity to SEQ ID NO:2; (iii) a sequence having at least 90% at least 91%, at least 92%, at least 93%, at least 94%, at least 95%, at least 96%, at least 97%, at least 98%, or at least 99% identity to SEQ ID NO:3; (iv) a sequence having at least 90% at least 91%, at least 92%, at least 93%, at least 94%, at least 95%, at least 96%, at least 97%, at least 98%, or at least 99% identity to SEQ ID NO:4; (v) a sequence having at least 90%, at least 91%, at least 92%, at least 93%, at least 94%, at least 95%, at least 96%, at least 97%, at least 98%, or at least 99% identity to SEQ ID NO:5; (vi) a sequence having at least 90%, at least 91%, at least 92%, at least 93%, at least 94%, at least 95%, at least 96%, at least 97%, at least 98%, or at least 99% identity to SEQ ID NO:6; (vii) a sequence having at least 90%, at least 91%, at least 92%, at least 93%, at least 94%, at least 95%, at least 96%, at least 97%, at least 98%, or at least 99% identity to SEQ ID NO:7; (viii) a sequence having at least 90%, at least 91%, at least 92%, at least 93%, at least 94%, at least 95%, at least 96%, at least 97%, at least 98%, or at least 99% identity to SEQ ID NO:8; (ix) a sequence having at least 90%, at least 91%, at least 92%, at least 93%, at least 94%, at least 95%, at least 96%, at least 97%, at least 98%, or at least 99% identity to SEQ ID NO:9; (x) a sequence having at least 90%, at least 91%, at least 92%, at least 93%, at least 94%, at least 95%, at least 96%, at least 97%, at least 98%, or at least 99% identity to SEQ ID NO:10; (xi) a sequence having at least 90%, at least 91%, at least 92%, at least 93%, at least 94%, at least 95%, at least 96%, at least 97%, at least 98%, or at least 99% identity to SEQ ID NO:11; (xii) a sequence having at least 90%, at least 91%, at least 92%, at least 93%, at least 94%, at least 95%, at least 96%, at least 97%, at least 98%, or at least 99% identity to SEQ ID NO: 12; (xiii) a sequence having at least 90%, at least 91%, at least 92%, at least 93%, at least 94%, at least 95%, at least 96%, at least 97%, at least 98%, or at least 99% identity to SEQ ID NO:13; (xiv) a sequence having at least 90%, at least 91%, at least 92%, at least 93%, at least 94%, at least 95%, at least 96%, at least 97%, at least 98%, or at least 99% identity to SEQ ID NO:14; (xv) a sequence having at least 90%, at least 91%, at least 92%, at least 93%, at least 94%, at least 95%, at least 96%, at least 97%, at least 98%, or at least 99% identity to SEQ ID NO:15; and (xvi) a sequence having at least 90%, at least 91%, at least 92%, at least 93%, at least 94%, at least 95%, at least 96%, at least 97%, at least 98%, or at least 99% identity to SEQ ID NO:16. In some aspects, the HTI immunogen is a nucleic acid encoding such a polypeptide.

In some aspects, the HTI immunogen is a polypeptide comprising (i) a sequence having at least 95% identity to SEQ ID NO:1; (ii) a sequence having at least 95% identity to SEQ ID NO:2; (iii) a sequence having at least 95% identity to SEQ ID NO:3; (iv) a sequence having at least 95% identity to SEQ ID NO:4; (v) a sequence having at least 95% identity to SEQ ID NO:5; (vi) a sequence having at least 95% identity to SEQ ID NO:6; (vii) a sequence having at least 95% identity to SEQ ID NO:7; (viii) a sequence having at least 95% identity to SEQ ID NO:8; (ix) a sequence having at least 95% identity to SEQ ID NO:9; (x) a sequence having at least 95% identity to SEQ ID NO:10; (xi) a sequence having at least 95% identity to SEQ ID NO:11; (xii) a sequence having at least 95% identity to SEQ ID NO:12; (xiii) a sequence having at least 95% identity to SEQ ID NO:13; (xiv) a sequence having at least 95% identity to SEQ ID NO: 14; (xv) a sequence having at least 95% identity to SEQ ID NO: 15; and (xvi) a sequence having at least 95% identity to SEQ ID NO:16. In some aspects, the HTI immunogen is a nucleic acid encoding such a polypeptide.

In some aspects, the HTI immunogen is a polypeptide comprising the sequences of SEQ ID NOs: 1-16. In some aspects, the HTI immunogen is a nucleic acid encoding such a polypeptide.

In some aspects, the HTI immunogen is a polypeptide comprising a sequence having at least 90%, at least 91%, at least 92%, at least 93%, at least 94%, at least 95%, at least 96%, at least 97%, at least 98%, or at least 99% identity to SEQ ID NO:99. In some aspects, the HTI immunogen is a nucleic acid encoding such a polypeptide.

In some aspects, the HTI immunogen is a polypeptide comprising a sequence having at least 95% identity to SEQ ID NO:99. In some aspects, the HTI immunogen is a nucleic acid encoding such a polypeptide.

In some aspects, the HTI immunogen is a polypeptide comprising the sequence of SEQ ID NO:99. In some aspects, the HTI immunogen is a nucleic acid encoding such a polypeptide.

In some aspects, one or more segments of the HTI immunogen (e.g., SEQ ID NOs: 1-16) are adjoined by an amino acid linker. In some aspects, two segments are adjoined, three segments are adjoined, four segments are adjoined, five segments are adjoined, six segments are adjoined, seven segments are adjoined, eight segments are adjoined, nine segments are adjoined, 10 segments are adjoined, 11 segments are adjoined, 12 segments are adjoined, 13 segments are adjoined, 14 segments are adjoined, 15 segments are adjoined or 16 segments are adjoined. In some aspects, the amino acid linker is a single (A), dual (AA), or triple (AAA) alanine linker. In some aspects, the amino acid linker results in the formation of an AAA sequence in the junction region between adjoining segments.

In some aspects, the HTI immunogen is a nucleic acid encoding any of the HTI immunogen polypeptides provided herein. In some aspects, the HTI immunogen is a nucleic acid comprising a sequence having at least 90%, at least 91%, at least 92%, at least 93%, at least 94%, at least 95%, at least 96%, at least 97%, at least 98%, or at least 99% identity to SEQ ID NO:100. In some aspects, the HTI immunogen is a polypeptide encoded by such a nucleic acid.

In some aspects, the HTI immunogen is a nucleic acid comprising a sequence having at least 95% identity to SEQ ID NO:100. In some aspects, the HTI immunogen is a polypeptide encoded by such a nucleic acid.

In some aspects, the HTI immunogen is a nucleic acid comprising the sequence of SEQ ID NO:100. In some aspects, the HTI immunogen is a polypeptide encoded by such a nucleic acid.

In some aspects, the HTI immunogen is administered to a subject as a vaccine (“HTI vaccine”). As used herein, the term “HTI vaccine” refers to the use of an HTI immunogen to provoke an immune response (e.g., produce neutralizing anti-HIV antisera). Accordingly, an HTI vaccine comprises an HTI immunogen. Administration of an HTI vaccine to a subject can confer at least partial immunity against HIV infection.

In some aspects, the HTI vaccine comprises an HTI immunogen in a vector. As used herein, the term “vector” refers to a nucleic acid used to introduce heterologous nucleic acids into a cell (e.g., an HTI immunogen) that has regulatory elements to provide expression of the heterologous nucleic acids in the cell. Examples of a vector include, but are not limited to, a plasmid, minicircle, yeast, and viral genome.

In some aspects, the vector is a viral vector. In some aspects, the viral vector is influenza, vesicular stomatitis virus, measles virus, or adenovirus. In some aspects, the viral vector comprises a poxvirus viral vector, e.g., a modified vaccinia virus Ankara (MVA) vector such as the MVA.HTI vector (see, e.g., U.S. Pub. No. 2022/0226459, U.S. Pat. No. 11,666,651, and U.S. Prov. Appl. No. 63/380,113, which are incorporated herein by reference in their entireties). An exemplary MVA vector is described in Barouch, D. H. et al. Cell 2013, 155 (3), 531-539 (incorporated herein by reference in its entirety). In some aspects, the viral vector comprises an adenovirus viral vector, such as a chimpanzee adenovirus, e.g., a replication-defective chimpanzee adenovirus. Exemplary chimpanzee adenovirus vectors have been described, e.g., in Int'l Pub. No. WO 2020/234839; Int'l Pub. No. WO 2021/094984; Int'l Pub. No. WO 2020/237027; U.S. Pub. No. 2022/0226459; U.S. Pat. Nos. 9,714,435, 11,666,651, and U.S. Prov. Appl. No. 63/380,113 (incorporated herein by reference in their entireties). In some aspects, the chimpanzee adenovirus vector is ChAdOx1.HTI (see, e.g., U.S. Pub. No. 2022/0226459, U.S. Pat. No. 11,666,651, and U.S. Prov. Appl. No. 63/380,113, which are incorporated herein by reference in their entireties).

By “immune response” is meant any response to an antigen or antigenic determinant by the immune system of a subject (e.g., a human). Exemplary immune responses include humoral immune responses (e.g., production of antigen-specific antibodies e.g., neutralizing antibodies (NAbs)) and cell mediated immune responses (e.g., lymphocyte proliferation). Exemplary immune responses also include those that can be determined by an immunoassay, as described further herein. In some aspects, an immune response is “elevated,” as described further herein.

A person skilled in the art will recognize multiple methods are available to measure an immune response to an HTI immunogen. Such methods include, but are not limited to, immunoassays (e.g., enzyme-linked immunosorbent (ELISA) assay (e.g., direct, indirect, competitive, or sandwich); enzyme-linked immunosorbent spot (ELISpot) assay (e.g., DC-ELISpot)); intracellular cytokine staining (ICS); flow cytometry, detection of neutralizing antibodies (e.g., seroneutralization, dot blot, immunoperoxidase monolayer assay (IPMA), ELISA (e.g., direct, indirect, competitive, bridging), or electrochemiluminiscence (e.g., with direct/indirect bridging format), radioimmunoprecipitation assay, surface plasmon resonance; cell-based bioassay, or competitive ligand binding assay); protein production assay (e.g., western blot, ELISA (e.g., direct, indirect or sandwich), ICS, flow cytometry, high-performance liquid chromatography (HPLC), or mass spectrometry); and/or RNA production assays (e.g., quantitative polymerase chain reaction (qPCR) or gene expression assays), among others. In some aspects, the evaluating the magnitude of the immune response to the HTI immunogen is done by an immunoassay. In some aspects, the immunoassay is an ELISpot assay. In some aspects, the immunoassay is an interferon-gamma (IFN-γ) ELISpot assay.

In some aspects, evaluating the magnitude of the immune response to the HTI immunogen includes collecting a biological sample from the subject. A person skilled in the art would understand examples of a biological sample and what methods could be used to obtain the same. In some aspects, the biological sample is, for example, blood, plasma, urine or saliva. In some aspects, the biological sample is blood. In some aspects, the biological sample is plasma. In some aspects, the biological sample is PBMC.

In some aspects, the evaluating comprises collecting a biological sample from the subject; and determining binding of the sample to an HTI antigen by immunoassay as a measurement of an immune response to the HTI immunogen

In some aspects, the evaluating comprises collecting a biological sample from the subject; contacting the sample with an ELISpot plate coated with anti-interferon-gamma (IFN-γ) antibodies; contacting the sample with an HTI antigen; and measuring the number of complexes (spot forming units or sfu) formed by the contacting.

In some aspects, evaluating the magnitude of the immune response to the HTI immunogen includes isolating PBMC from blood plasma, plating the isolated PBMC on an IFN-γ monoclonal antibody-coated ELISpot plate, contacting the PBMC with an HTI antigen, and measuring the number of spots formed by the HTI-stimulated PBMC in the sample. In some aspects, if the number of spots formed reaches a certain threshold above the background and controls, the antiretroviral treatment can be stopped, and the HTI immune response induced by the vaccine will keep the HIV below acceptable levels.

In some aspects, an acceptable HIV level is less than 10,000 copies of virus/mL, preferably less than 1,000 copies of virus/mL, more preferably less than 200 copies of virus/mL, and more preferably undetectable levels of virus/mL of blood and/or a CD4 level of from about 300 to about 500 cells/mm³ in blood.

In some aspects, the methods provided herein refer to an “elevated” immune response to the HTI immunogen. As used herein, this term refers to an immune response that is greater than a threshold value. In some aspects, the elevated immune response is determined relative to a threshold value. In some aspects, the threshold value is a control value from a subject wherein the HTI immunogen is not administered.

In some aspects, the elevated immune response is determined relative to a threshold value. In some aspects, the threshold value is 300 or more, 400 or more, 500 or more, 600 or more, 700 or more, 800 or more, 900 or more, 1,000 or more, 1,100 or more, 1,200 or more, 1,300 or more, 1,400 or more, 1,500 or more, 1,600 or more, 1,700 or more, 1,800 or more, 1,900 or more, or 2,000 or more spot forming units (sfu) against the HTI antigen per million PBMC.

In some aspects, the threshold value is about 300, about 400, about 500, about 600, about 700, about 800, about 900, about 1,000, about 1,100, about 1,200, about 1,300, about 1,400, about 1,500, about 1,600, about 1,700, about 1,800, about 1,900, or about 2,000 sfu against the HTI antigen per million PBMC.

In some aspects, the threshold value is from about 300 to about 2,000 sfu against the HTI antigen per million PBMC, or any values or range of values thereof (e.g., from about 300 to about 1,000, from about 500 to about 1,000, from about 700 to about 1,000).

In some aspects, the HIV is HIV-1. In some aspects, the HIV is HIV-2.

In some aspects, the methods of the present disclosure pertain to antiretroviral therapy. As used herein, the terms “antiretroviral therapy” or “ART” refer to treatment of a subject infected with HIV using anti-HIV drugs. A person skilled in the art would readily understand examples of antiretroviral therapies and how to administer the same to a subject. (Bean, B., Clin. Microbiol. Rev., 5 (2): 146-182, 1992; and Kausar et al., Int'l J. Immunopathol. Pharmacol. 35:1-12, 2021; for example). In some aspects, the antiretroviral therapy is a nucleoside reverse transcriptase inhibitor (NRTI; e.g., lamivudine, abacavir, didanosine, tenofovir disoprovil fumarate, emtricitabine, stavudine, zalcitabine, zidovudine, or a combination thereof); a non-nucleotide reverse transcriptase inhibitor (NNRTI; e.g., efavirenz, etravirine, rilpivirine, doravirine, or a combination thereof); a protease inhibitor (atazanavir, darunavir, or a combination thereof); an entry inhibitor (e.g., maraviroc, ibalizumab-uiyk, fostemsavir, or a combination thereof); an integrase inhibitor (e.g., raltegravir, dolutegravir, elvitegravir, cabotegravir, or a combination thereof); a pharmacokinetic enhancer (e.g., ritonavir, cobicistat, or a combination thereof); or a combination thereof.

In some aspects, the methods, compositions and kits provided herein incorporate an HTI antigen. As used herein, the term “HTI antigen” refers to the HTI immunogen or any fragment or derivative thereof that induces an immune response. Examples of an HTI antigen include, but are not limited to, one or more overlapping peptides (OLPs) covering the HTI immunogen (e.g., as listed in Table 1). Accordingly, in some aspects, an HTI antigen has the amino acid sequence of any one of SEQ ID NOs: 101-211, or any combination thereof, or an amino acid sequence at least 90%, at least 91%, at least 92%, at least 93%, at least 94%, at least 95%, at least 96%, at least 97%, at least 98%, or at least 99% identical to any one of SEQ ID NOs: 101-211, or any combination thereof.

In some aspects, the evaluating of the magnitude of the subject's immune response to the HTI immunogen is done at or after about one week, about two weeks, about three weeks, about four weeks, about five weeks, about six weeks, about seven weeks, or about eight weeks after administering the HTI immunogen. In some aspects, the evaluating of the magnitude of the subject's immune response to the HTI immunogen is done at about the time of administering the HTI immunogen.

In some aspects, the evaluating of the magnitude of the subject's immune response to the HTI immunogen is done at or after about one month, about two months, about three months, about four months, about five months, about six months, about seven months, about eight months, about nine months, about 10 months, about 11 months, or about 12 months after administering the HTI immunogen.

In some aspects, the evaluating of the magnitude of the subject's immune response to the HTI immunogen is done at or after about one year, about two years, about three years, about four years, or about five years after administering the HTI immunogen.

In some aspects, the immune response corresponds to the magnitude of HTI-specific T-cells of the subject at a given timepoint during the administering of the HTI immunogen. In some aspects, the immune response corresponds to magnitude of HTI-specific T-cells of the subject any time after the administering of the HTI immunogen (e.g., at about one week, about two weeks, about three weeks, about four weeks, about five weeks, or about six weeks).

In some aspects, the immune response corresponds to the magnitude of HTI-specific T-cells of the subject at from about four to about 12 weeks after the administering of the HTI immunogen. In some aspects, the immune response corresponds to magnitude of HTI-specific T-cells of the subject at about four, about five, about six, about seven, about eight, about nine, about 10, about 11, or about 12 weeks after the administering of the HTI immunogen. In some aspects, the immune response corresponds to magnitude of HTI-specific T-cells of the subject at about eight weeks after the administering of the HTI immunogen.

In some aspects, the immune response allows the subject to be stratified by vaccination outcome according to responders and non-responders. In some aspects, the subject is stratified using a threshold value. In some aspects, the threshold value is the maximum HTI-magnitude of the subject. In some aspects, the maximum HTI-magnitude is from about 500 SFC/106 to about 2,000 SFC/106. In some aspects, the maximum HTI-magnitude is about 1,000 SFC/106.

The level of immune response to HTI used to stratify the outcome to the vaccination in high responders and low responders by using a threshold is set in clinical studies. The level of immune response can be set in absolute terms, for example, above a certain level of immune response in an ELISpot assay, or it can be used in reference to a certain patient population, for example as above or below a threshold in the distribution of the immune responses, for example above the median of the immune responses against HTI.

In some aspects, the method includes obtaining a blood sample from a human subject, preferably a subject living with HIV; and isolating PBMC from the sample to assess the immune response to the HTI immunogen.

In some aspects, the immune response to the HTI immunogen can be measured at certain times, including, but not limited to, immediately before and after the first vaccination with the HTI immunogen, at multiple points during an HTI vaccination regime, and at multiple points before and after the interruption of antiretroviral therapy.

In some aspects, the level of immune response to HTI used for the method of the present invention includes the immune response to HTI before the vaccination, the peak immune response at any time after vaccination, and the immune response before the decision to interrupt the antiretroviral treatment is made.

In some aspects, the method to determine the probability of HIV control in the treatment decision can include the combination of the magnitude of the immune response against HTI with additional biomarkers including, but not limited to, breadth of the immune response against HTI, avidity of the immune response against HTI, type of immune responses against HTI, antibodies against HTI, types of immune cells (related with both natural and adaptive immune responses), level of cytokines and T-cell biomarkers, HLA typing of the subject, viral load prior to the first administration of ART, time on ART prior to vaccination, viral reservoir at ATI start, and other immune-related mediators.

In some aspects, the magnitude of the immune response against HTI can be used in multiple settings not including a prior HTI vaccination, to determine the probability of HIV control in the decision to administer or not antiretroviral medication. For example, measuring the magnitude of the immune response against HTI can be used to decide if a patient should start antiretrovirals or not for the first time, or after a pause, or it can be used to decide if a patient can interrupt antiretrovirals after a vaccination regime with another vaccine that does not include the HTI vaccine.

In some aspects, the magnitude of the immune response against HTI can be used in the preventive setting, i.e., after vaccination with HTI (or with any other HIV vaccine) of the general population not infected with HIV, as a preventive measure. In this case, the magnitude of the immune response against HTI can be used as surrogate for vaccine efficacy and for the level of protection, for example indicating the need of additional vaccine boosters.

The present disclosure also relates to an HTI antigen for evaluating magnitude of an immune response in a biological sample. In some aspects, the biological sample is blood. In some aspects, the biological sample is PBMC.

The present disclosure also relates to an HTI antigen for identifying a human subject infected with HIV that can stop treatment with an antiretroviral therapy.

The present disclosure also relates to an HTI antigen for identifying a human subject infected with HIV that can continue treatment with an antiretroviral therapy.

The present disclosure also relates to an HTI antigen for identifying a human subject infected with HIV that can start treatment with an antiretroviral therapy.

The present disclosure also relates to a composition comprising an HTI antigen provided herein and a pharmaceutically acceptable carrier. Examples of a pharmaceutically acceptable carrier are known and include, but are not limited to, a solid, semisolid or liquid filler, diluent, encapsulating material or formulation auxiliary of any conventional type. Other suitable pharmaceutically acceptable carriers include, but are not limited to, water, dextrose, glycerol, saline, ethanol, and combinations thereof. In some aspects, a pharmaceutically acceptable carrier can contain additional agents such as wetting or emulsifying agents, pH buffering agents, or adjuvants that enhance the effectiveness of the formulation

The present disclosure also relates to a kit comprising an HTI antigen provided herein and instructions for use in any of the methods provided herein.

In some aspects, the kit provides for the selection of patients eligible to complete treatment of HIV infection by safely interrupting antiretroviral therapy. In some aspects, the kit provides for the selection of eligible patients by quantifying in a biological sample the level of immune response to an HTI immunogen. In some aspects, the biological sample is PBMC.

### EXAMPLES

Reference is now made to the following example, which together with the above description illustrates some aspects in a non-limiting fashion.

### Example 1

Virologic Control of HTI-Vaccinated Subjects Living with HIV Measured as ART Since ATI

A clinical study was performed to evaluate virologic control of subjects living with human immunodeficiency virus (HIV). Subjects were vaccinated with HIVACAT T-cell immunogen (HTI), the immunogenic polypeptide having an amino acid sequence of SEQ ID NOs: 1-16, as follows.

### Vaccine Administration and Sampling Schedule

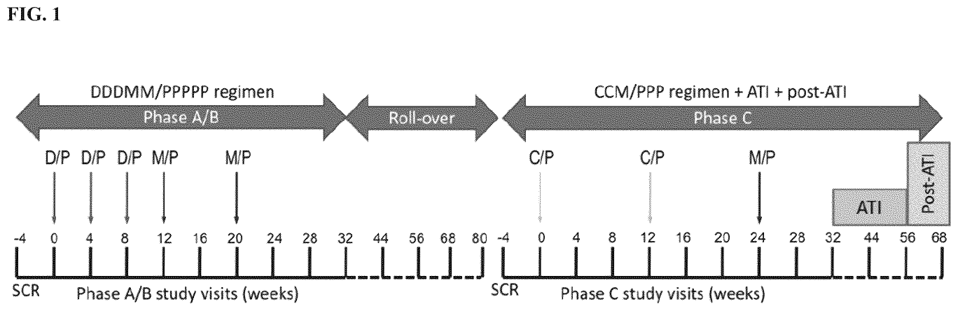

The study was divided into two phases, Phase A/B and Phase C, with a “Roll-over” phase of variable extension between the two phases (FIG. 1). The first phase, Phase A/B, consisted of administration of (a) the heterologous “DDDMM” regimen [wherein D=administration of HTI in a DNA vector (DNA.HTI), and M=administration of HTI in a Modified Vaccinia Ankara (MVA) vector (MVA.HTI)], or (b) a placebo (P) regimen (“PPPPP”). D was administered intramuscularly (IM) at a dose of 4 mg. M was administered IM at a dose of 2×10⁸ plaque-forming units (pfu).

The second phase, Phase C, consisted of administration of (a) the heterologous “CCM” regimen [wherein C=administration of HTI in the ChAdOx adenovirus vector (ChAdOx1.HTI), and M is as above], of (b) a placebo (P) regimen (“PPP”). C was administered IM at a dose of 5×10¹⁰ viral particles (Vp).

The second phase was then followed by an analytical treatment interruption (ATI), and a post-ATI follow-up period after antiretroviral therapy (ART) resumption. The intervention started on the day of the first DNA.HTI/placebo administration, which was considered Week 0 of Phase A/B. And, the study ended after 12 weeks of post-ATI follow-up, at Week 68 of Phase C.

Study participants were included in two recruitment periods. Fifteen participants were included in Phase A, and thirty participants were included in Phase B. All participants followed the same vaccination regimen.

Blood samples were collected from study participants at Screening, Week 0, Week 8, Week 9, Week 12, Week 13, Week 20, Week 21, Week 22, Week 24 and Week 32 of Phase A/B; at Week 44, Week 56, Week 68 and Week 80 of the Roll-over phase; and at Screening, Week 0, Week 4, Week 12, Week 16, Week 24, Week 28 and Week 32 (ATI start) of Phase C. After collection, peripheral blood mononuclear cells (PBMC) were isolated from the blood samples and tested, as described below.

### HTI Response Measurement Using IFN-γ ELISpot

For HTI response measurement, blood samples from study participants were obtained using 10 mL K2E 18 mg (EDTA) tubes. Blood plasma and PBMC were isolated within 3 hours after blood draws. Freshly isolated PBMC were retrieved and counted to assess number and viability of cells. Cell suspensions were incubated at 37° C. and 5% CO₂ for 18-20 hours, after which cell viability and viable cell count were again determined. Where the yield of cells was sufficient, cells were plated at 100,000 cells/well on an interferon-gamma (IFN-γ) monoclonal antibody-coated enzyme-linked immunosorbent spot (ELISpot) plate and stimulated with HTI antigens at 37° C. and 5% CO₂ for 24 hours. Following stimulation, cell suspensions were removed, plates were washed 5 times with phosphate buffered saline (PBS), biotinylated detection secondary antibody was added to all wells, and plates were incubated for 2 hours to 2 hours and 15 minutes. The secondary antibody was removed, the plates were washed 5 times with PBS, and the enzyme, streptavidin alkaline phosphatase (ALP), was added to all wells and incubated for 1 hour to 1 hour and 15 minutes. Streptavidin ALP was then removed and the plates were washed five times with PBS. The enzyme substrate, NBT/BCIP (nitro blue tetrazolium/5-bromo-4-chloro-3-indolyl phosphate) was then added to all wells and the plates were incubated for 3 minutes. Spot color development was increased by submerging plates into water. Developed plates were left to dry (protected from light) at least overnight. Colored spots on developed plates were scanned and automatically counted. Positive controls (incubation with phytohemaglutin, PHA) and negative controls (incubation with dimethyl sulfoxide, DMSO) were also used.

### Biomarker Calculation and Subject Stratification

Response to HTI immunogen for a given subject at a given timepoint was reported in Spot Forming Counts (SFC) per million PBMC (SFC/106 PBMC). Each sample was associated to a cutoff value to assess positivity of the response. Negative well counts were used to set the cutoff value. The cutoff value was the resulting of the maximum value from either (1) 50 SFC/106 PBMCs; (2) the average of the negative control wells counts multiplied by 3; or (3) the average of the negative control well counts plus 3 standard deviations.

The magnitude of Total HTI-specific T-cells of a sample or at a given timepoint was calculated and the magnitude of Total HTI-specific response at ATI start (Week 32 of phase C) was used to (1) calculate the median magnitude of Total HTI-specific response of the study population (threshold); and (2) to stratify study participants into a High/Low HTI subgroup based on the threshold value calculated.

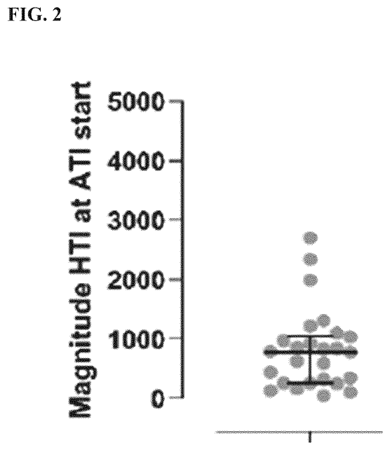

Median magnitude of Total-HTI-specific T-cells at ATI start was 790 SPC/10⁶ PBMC, ranging from 50 to 2.655 (FIG. 2).

### HTI-Specific Peptide Pools and Out Peptide Pools

HTI-specific T-cell data comprises data from 10 peptide pools covering the HTI immunogen, distributed from pools HTI-p1 to HTI-p10. Composition of the pools is detailed in Table 1.

TABLE 1
Exemplary HTI Antigens

| HIV | | | | |
|---|---|---|---|---|
| protein | Subprotein | Amino Acid Sequence | Pool Name | SEQ ID NO: |
| Gag | p17 | VEKIRLRPGGKKKYK | HTI-p1 (p17) | 101 |
| Gag | p17 | RLRPGGKKKYKLKHI | HTI-p1 (p17) | 102 |
| Gag | p17 | GGKKKYKLKHIVWA | HTI-p1 (p17) | 103 |
| Gag | p17 | KKYKLKHIVWASREL | HTI-p1 (p17) | 104 |
| Gag | p17 | LKHIVWASRELERFA | HTI-p1 (p17) | 105 |
| Gag | p17 | VWASRELERFAV | HTI-p1 (p17) | 106 |
| Gag | p17 | WASRELERFAVNPGL | HTI-p1 (p17) | 107 |
| Gag | p17 | ELERFAVNPGLL | HTI-p1 (p17) | 108 |
| Gag | p17 | LERFAVNPGLLETSE | HTI-p1 (p17) | 109 |
| Gag | p17 | AVNPGLLETSEGCR | HTI-p1 (p17) | 110 |
| Gag | p17 | PGLLETSEGCRQIL | HTI-p1 (p17) | 111 |
| Gag | p17 | LETSEGCRQILGQL | HTI-p2 (p17) | 112 |
| Gag | p17 | SEGCRQILGQLQPSL | HTI-p2 (p17) | 113 |
| Gag | p17 | RQILGQLQPSLQTGS | HTI-p2 (p17) | 114 |
| Gag | p17 | GQLQPSLQTGSEELK | HTI-p2 (p17) | 115 |
| Gag | p17 | PSLQTGSEELKSLY | HTI-p2 (p17) | 116 |
| Gag | p17 | QTGSEELKSLYNTVA | HTI-p2 (p17) | 117 |
| Gag | p17 | EELKSLYNTVATLY | HTI-p2 (p17) | 118 |
| Gag | p17 | KSLYNTVATLYCVH | HTI-p2 (p17) | 119 |
| Gag | p17 | YNTVATLYCVHQKI | HTI-p2 (p17) | 120 |
| Gag | p17 | VATLYCVHQKIEVAA | HTI-p2 (p17) | 121 |
| Gag | p24 | KAFSPEVIPMESALA | HTI-p3 (p24) | 122 |
| Gag | p24 | AGHQAAMQMLKEAAA | HTI-p3 (p24) | 123 |
| Gag | p24 | AIAPGQMREPRGSDI | HTI-p3 (p24) | 124 |
| Gag | p24 | GQMREPRGSDIA | HTI-p3 (p24) | 125 |
| Gag | p24 | QMREPRGSDIAGTTSTL | HTI-p3 (p24) | 126 |
| Gag | p24 | GSDIAGTTSTLQEQI | HTI-p3 (p24) | 127 |
| Gag | p24 | AGTTSTLQEQIGWM | HTI-p3 (p24) | 128 |
| Gag | p24 | TSTLQEQIGWMTNNPPI | HTI-p3 (p24) | 129 |
| Gag | p24 | QIGWMTNNPPIPV | HTI-p3 (p24) | 130 |
| Gag | p24 | GWMTNNPPIPVGEIY | HTI-p4 (p24) | 131 |
| Gag | p24 | NNPPIPVGEIYKRWI | HTI-p4 (p24) | 132 |
| Gag | p24 | IPVGEIYKRWIILGL | HTI-p4 (p24) | 133 |
| Gag | p24 | EIYKRWIILGLNKIV | HTI-p4 (p24) | 134 |
| Gag | p24 | RWIILGLNKIVRMY | HTI-p4 (p24) | 135 |
| Gag | p24 | ILGLNKIVRMYSPTSI | HTI-p4 (p24) | 136 |
| Gag | p24 | YVDRFYKTLRAEQAA | HTI-p4 (p24) | 137 |
| Gag | p24 | AACQGVGGPGHKARV | HTI-p4 (p24) | 138 |
| Gag | p24 | GVGGPGHKARVLAAA | HTI-p4 (p24) | 139 |
| Gag | p2p7p1p6 | AAACTERQANFLGKI | HTI-p5 (p15) | 140 |
| Gag | p2p7p1p6 | TERQANFLGKIWPSH | HTI-p5 (p15) | 141 |
| Gag | p2p7p1p6 | ANFLGKIWPSHKGR | HTI-p5 (p15) | 142 |
| Gag | p2p7p1p6 | LGKIWPSHKGRPGNF | HTI-p5 (p15) | 143 |
| Gag | p2p7p1p6 | WPSHKGRPGNFLQSR | HTI-p5 (p15) | 144 |
| Pol | Prot | AKMIGGIGGFIKVR | HTI-p6 (prot) | 145 |
| Pol | Prot | IGGIGGFIKVRQY | HTI-p6 (prot) | 146 |
| Pol | Prot | GIGGFIKVRQYDQIL | HTI-p6 (prot) | 147 |
| Pol | Prot | FIKVRQYDQILIEI | HTI-p6 (prot) | 148 |
| Pol | Prot | VRQYDQILIEICGHK | HTI-p6 (prot) | 149 |
| Pol | Prot | DQILIEICGHKAI | HTI-p6 (prot) | 150 |
| Pol | Prot | ILIEICGHKAIGTVL | HTI-p6 (prot) | 151 |
| Pol | Prot | ICGHKAIGTVLV | HTI-p6 (prot) | 152 |
| Pol | Prot | CGHKAIGTVLVGPTPV | HTI-p6 (prot) | 153 |
| Pol | Prot | IGTVLVGPTPVNII | HTI-p6 (prot) | 154 |
| Pol | Prot | VLVGPTPVNIIGRNL | HTI-p6 (prot) | 155 |
| Pol | Prot | PTPVNIIGRNLLTQI | HTI-p6 (prot) | 156 |
| Pol | Prot | NIIGRNLLTQIGCTL | HTI-p6 (prot) | 157 |
| Pol | Prot | RNLLTQIGCTLNFAA | HTI-p6 (prot) | 158 |
| Pol | RT | ALVEICTEMEKEGKI | HTI-p7 (RT) | 159 |
| Pol | RT | ICTEMEKEGKISKIA | HTI-p7 (RT) | 160 |
| Pol | RT | ALRWGFTTPDKKHQK | HTI-p7 (RT) | 161 |
| Pol | RT | GFTTPDKKHQKEPPF | HTI-p7 (RT) | 162 |
| Pol | RT | PDKKHQKEPPFLWM | HTI-p7 (RT) | 163 |
| Pol | RT | KHQKEPPFLWMGYEL | HTI-p7 (RT) | 164 |
| Pol | RT | EPPFLWMGYELHPDK | HTI-p7 (RT) | 165 |
| Pol | RT | LWMGYELHPDKWTV | HTI-p7 (RT) | 166 |
| Pol | RT | GYELHPDKWTVQPIV | HTI-p7 (RT) | 167 |
| Pol | RT | HPDKWTVQPIVLPEK | HTI-p7 (RT) | 168 |
| Pol | RT | WTVQPIVLPEKDSW | HTI-p7 (RT) | 169 |
| Pol | RT | QPIVLPEKDSWTV | HTI-p7 (RT) | 170 |
| Pol | RT | IVLPEKDSWTVNDI | HTI-p7 (RT) | 171 |
| Pol | RT | PEKDSWTVNDIQKLV | HTI-p7 (RT) | 172 |
| Pol | RT | SWTVNDIQKLVGKLA | HTI-p7 (RT) | 173 |
| Pol | RT | AILKEPVHGVYY | HTI-p7 (RT) | 174 |
| Pol | RT | ILKEPVHGVYYDPSK | HTI-p7 (RT) | 175 |
| Pol | RT | PVHGVYYDPSKDLIA | HTI-p7 (RT) | 176 |
| Pol | RT | VYYDPSKDLIAEIQK | HTI-p7 (RT) | 177 |
| Pol | RT | PSKDLIAEIQKQGQGQW | HTI-p7 (RT) | 178 |
| Pol | RT | AEIQKQGQGQWTYQI | HTI-p7 (RT) | 179 |
| Pol | RT | KQGQGQWTYQIYAAA | HTI-p7 (RT) | 180 |
| Pol | Int | TKELQKQITKIQNFR | HTI-p8 (Int) | 181 |
| Pol | Int | QKQITKIQNFRVYYR | HTI-p8 (Int) | 182 |
| Pol | Int | TKIQNFRVYYRDSR | HTI-p8 (Int) | 183 |
| Pol | Int | QNFRVYYRDSRDPLW | HTI-p8 (Int) | 184 |
| Pol | Int | VYYRDSRDPLWKGPA | HTI-p8 (Int) | 185 |
| Pol | Int | DSRDPLWKGPAKLLW | HTI-p8 (Int) | 186 |
| Pol | Int | KIIRDYGKQMAGDDCV | HTI-p8 (Int) | 187 |
| Vif | Vif | AAVKHHMYISKKAK | HTI-p9 (Vif-Nef) | 188 |
| Vif | Vif | KHHMYISKKAKGWFY | HTI-p9 (Vif-Nef) | 189 |
| Vif | Vif | YISKKAKGWFYRHHY | HTI-p9 (Vif-Nef) | 190 |
| Vif | Vif | KAKGWFYRHHYESTH | HTI-p9 (Vif-Nef) | 191 |
| Vif | Vif | WFYRHHYESTHPRAA | HTI-p9 (Vif-Nef) | 192 |
| Vif | Vif | VTKLTEDRWNKPQK | HTI-p9 (Vif-Nef) | 193 |
| Vif | Vif | LTEDRWNKPQKTKGH | HTI-p9 (Vif-Nef) | 194 |
| Vif | Vif | RWNKPQKTKGHRAAA | HTI-p9 (Vif-Nef) | 195 |
| Nef | Nef | AWLEAQEEEEVGF | HTI-p9 (Vif-Nef) | 196 |
| Gag- | p24 | HQKIEVAAAKAFSPE | HTI-p10 (Linkers) | 197 |
| Linker | | | | |
| Gag- | p24 | PMFSALAAAGHQAAM | HTI-p10 (Linkers) | 198 |
| Linker | | | | |
| Gag- | p24 | MQMLKEAAAIAPGQM | HTI-p10 (Linkers) | 199 |
| Linker | | | | |
| Gag- | p24 | YSPTSIAAAYVDRFT | HTI-p10 (Linkers) | 200 |
| Linker | | | | |
| Gag- | p24 | LRAEQAAACQGVG | HTI-p10 (Linkers) | 201 |
| Linker | | | | |
| Gag- | p24 | HKARVLAAACTERQA | HTI-p10 (Linkers) | 202 |
| Linker | | | | |
| Pol-Linker | Prot | NFLQSRAAAKMIGGI | HTI-p10 (Linkers) | 203 |
| Pol-Linker | RT | GCTLNFAALVEICT | HTI-p10 (Linkers) | 204 |
| Pol-Linker | RT | GKISKIAAALRWGFT | HTI-p10 (Linkers) | 205 |
| Pol-Linker | RT | KLVGKLAAAILKEPV | HTI-p10 (Linkers) | 206 |
| Pol-Linker | Int | WTYQIYAAATKELQK | HTI-p10 (Linkers) | 207 |
| Pol-Linker | Int | PAKLLWAAAKIIRDY | HTI-p10 (Linkers) | 208 |
| Pol-Linker | Int | AGDDCVAAAVKHHMY | HTI-p10 (Linkers) | 209 |
| Vif-Linker | Vif | ESTHPRAAAVTKLTE | HTI-p10 (Linkers) | 210 |
| Nef- | Nef | KTKGHRAAAWLEAQE | HTI-p10 (Linkers) | 211 |
| Linker | | | | |

Non-HTI T-cell data comprises data from eight peptide pools distributed from pools OUT-p1 to OUT-p10. Composition of the pools is detailed in Table 1.

### Magnitude of Total HTI-Specific T-Cells

The magnitude of Total HTI-specific T-cells at a given timepoint is the sum of counts computed over the 10 pools HTI-p1 to HTI-p10. A response for a given subject, at a given timepoint is defined to be positive if the well count is above the cut-off value determined for that subject at that timepoint (unit is SFC/million PBMC). Individual counts below the positive response cut-off threshold are not taken into account for the sum of the magnitude of total HTI.

### Magnitude of Non-HTI T-Cells

The variable magnitude of non-HTI response at a given timepoint is the sum of counts computed over the 8 pools OUT-p1 to OUT-p8. Individual counts below the positive response cut-off threshold are not taken into account for the sum of magnitude.

### Focus of HIV-Specific Response

The focus of HTI-specific response at a given timepoint was defined as the magnitude of total HTI-specific T cells divided by the magnitude of the total HIV-1 proteome-specific T cells (magnitude of HTI-specific T cells+magnitude of non-HTI specific responses). The focus of HTI-specific response was expressed as a percentage.

### Breadth and Cumulative Breadth of Total HTI-Specific T-Cells

Breadth of total HTI-specific cells at a given timepoint was defined as the number of reactive pools (above the cut-off value) among the 10 HTI-specific peptide pools, with a range of 0-10 (due to the total number of HTI pools included in the ELISpot screens, count of positive HTI pools).

Cumulative breadth of total HTI-specific cells at a given timepoint was defined as the total number of distinct pools among the 10 HTI-specific peptide pools which have been reactive after baseline (above cut-off value) and up to the specific timepoint with a range of 0-10.

### Correlation of Magnitude Total HTI-Specific T-Cells and ATI Outcome

Correlations between HTI response and viral suppressive capacity measured as the time to ART resumption in ATI were assessed in all participants and the subgroup of participants without beneficial HLA class I alleles.

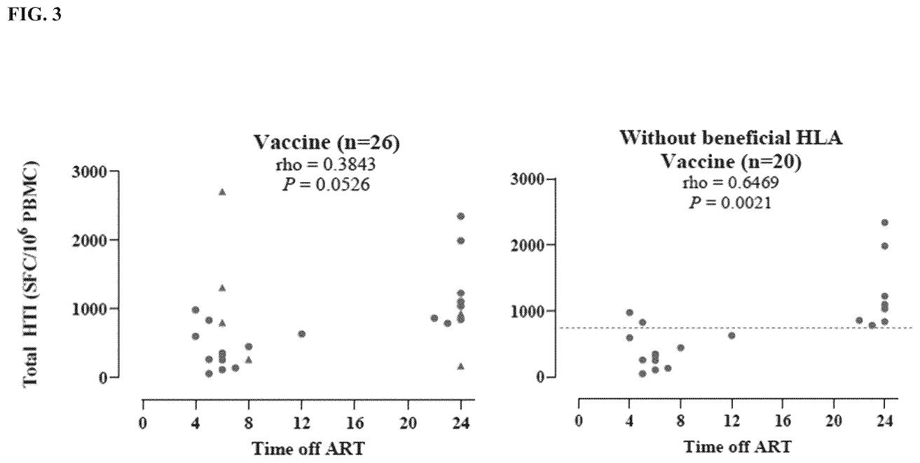

Vaccine recipients without beneficial HLA class I alleles showed positive and significant correlations with HTI magnitude, indicating that intensified HTI responses resulted in prolonged time off ART. Importantly, the magnitude of HTI-specific T-cell responses at ATI start and time off ART showed the strongest correlation, indicating a role of HTI responses in controlling viral rebound during ATI (FIG. 3).

### Kaplan-Meier Curves Between Participants and Time Off ART

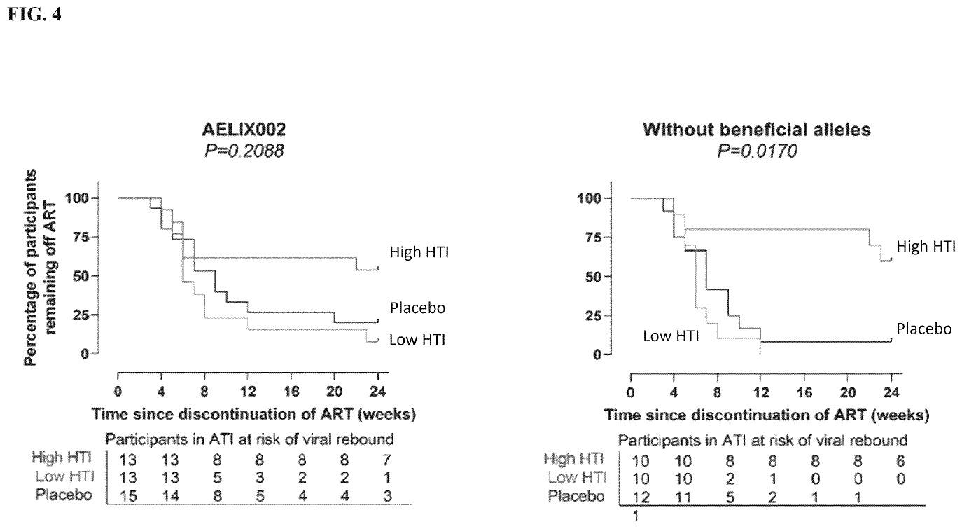

Survival curves were made by stratifying the vaccine recipients into high-HTI (above the median) and low-HTI (below the median) participants applying the threshold value of the median (790 SFC/106 PBMCs) and placebo recipients (FIG. 4). High-HTI vaccine recipients without beneficial HLA class I alleles showed a statistically significant probability of long term viral control compared to those whose response to HTI was low or placebo participants.

All publications, patents and patent applications mentioned in this application are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of treating a human immunodeficiency (HIV) infection in a human subject undergoing treatment with an antiretroviral therapy, comprising:
(a) administering a HIVACAT T-cell immunogen (HTI) immunogen to the subject;
(b) evaluating magnitude of the subject's immune response to the HTI immunogen; and
(c) stopping treatment with the antiretroviral therapy if the subject has an elevated immune response to the HTI immunogen.

2.-7. (canceled)

8. The method of claim 1, wherein the HTI immunogen is a polypeptide comprising, or a nucleic acid encoding a polypeptide comprising:
(i) a sequence having at least 95% identity to SEQ ID NO:1;
(ii) a sequence having at least 95% identity to SEQ ID NO:2;
(iii) a sequence having at least 95% identity to SEQ ID NO:3;
(iv) a sequence having at least 95% identity to SEQ ID NO:4;
(v) a sequence having at least 95% identity to SEQ ID NO:5;
(vi) a sequence having at least 95% identity to SEQ ID NO:6;
(vii) a sequence having at least 95% identity to SEQ ID NO:7;
(viii) a sequence having at least 95% identity to SEQ ID NO:8;
(ix) a sequence having at least 95% identity to SEQ ID NO:9;
(x) a sequence having at least 95% identity to SEQ ID NO:10;
(xi) a sequence having at least 95% identity to SEQ ID NO:11;
(xii) a sequence having at least 95% identity to SEQ ID NO:12;
(xiii) a sequence having at least 95% identity to SEQ ID NO:13;
(xiv) a sequence having at least 95% identity to SEQ ID NO: 14;
(xv) a sequence having at least 95% identity to SEQ ID NO:15; and
(xvi) a sequence having at least 95% identity to SEQ ID NO:16.

9.-10. (canceled)

11. The method of claim 1, wherein the HTI immunogen is a polypeptide comprising, or a nucleic acid encoding a polypeptide comprising a sequence having at least 90% identity to SEQ ID NO:99.

12.-13. (canceled)

14. The method of claim 1, wherein the sequences of (i)-(xvi) are adjoined by amino acid linkers.

15. The method of claim 14, wherein the amino acid linkers are single, dual, or triple alanine linkers, and wherein the linkers result in the formation of an AAA sequence in the junction region between adjoining sequences.

16. The method of claim 1, wherein the HTI immunogen is administered as a nucleic acid encoding the HTI immunogen.

17. The method of claim 16, wherein the nucleic acid comprises a sequence having at least 90% identity to SEQ ID NO:100.

18.-19. (canceled)

20. The method of claim 1, wherein the HTI immunogen is administered in a viral vector comprising a nucleic acid encoding the HTI immunogen.

21. The method of claim 20, wherein the viral vector is a Modified Vaccinia Ankara (MVA) vector, or a Chimpanzee adenovirus (ChAd) vector.

22. The method of claim 1, wherein the HTI immunogen is administered in combination with one or more antibodies, immunomodulators or other vaccines.

23. The method of claim 1, wherein the evaluating is done by an immunoassay.

24. The method of claim 23, wherein the immunoassay is an enzyme-linked immunosorbent spot (ELISpot) assay.

25. The method of claim 24, wherein the ELISpot assay is an interferon-gamma (IFN-γ) ELISpot assay.

26. The method of claim 1, wherein the evaluating comprises:
collecting a biological sample from the subject; and
determining binding of the sample to an HTI antigen by immunoassay as a measurement of an immune response to the HTI immunogen.

27. (canceled)

28. The method of claim 26, wherein biological sample is blood.

29. The method of claim 26, wherein the biological sample is peripheral blood mononuclear cells (PBMC).

30. The method of claim 1, wherein the HTI antigen has the amino acid sequence of any one of SEQ ID NOs: 101-211, or any combination thereof, or an amino acid sequence at least 90%, at least 91%, at least 92%, at least 93%, at least 94%, at least 95%, at least 96%, at least 97%, at least 98%, or at least 99% identical to any one of SEQ ID NOs: 101-211, or any combination thereof.

31.-61. (canceled)
